(19) United States
(12) Patent Application Publication
(10) Pub. No.: US 2013/0165809 A1
(43) Pub. Date: Jun. 27, 2013

(54) MONITORING PHYSIOLOGICAL CONDITION OF A SUBJECT

(71) Applicant: Eyall Abir, Petah Tikva (IL)

(72) Inventor: Eyall Abir, Petah Tikva (IL)

(73) Assignee: DIGISENSE LTD., Petach Tikva (IL)

(21) Appl. No.: 13/812,761

(22) PCT Filed: Jul. 28, 2011
(86) PCT No.: PCT/IL2011/000615

§ 371 Date: Mar. 11, 2013

Related U.S. Application Data
(60) Provisional application No. 61/368,654, filed on Jul. 29, 2010.

(51) Int. Cl. A61B 5/113; A61B 5/11; A61B 5/00

(52) U.S. Cl. CPC A61B 5/113; A61B 5/0004; A61B 5/746; A61B 5/7282; A61B 5/6808; A61B 5/0026; A61B 5/7455; A61B 5/7475; A61B 5/1126
USPC 600/534

(57) ABSTRACT

A system for monitoring breathing of a subject, the system comprising: (a) a wearable subject unit comprising: an elastic resistive sensor positionable such that breathing motion of the subject applies mechanical pressure to said elastic resistive sensor, wherein said elastic resistive sensor is configured to change its resistance responsive to the mechanical pressure, and a transmitter configured to wirelessly transmit a signal based on the change in resistance of the elastic resistive sensor; and (b) a platform unit comprising a receiver and being positionable in wireless transmission range with said subject unit, said platform unit configured to receive said signal from said subject unit and to issue an advisory signal indicative of the breathing of the subject.

### FIELD OF THE INVENTION

The invention relates to system and methods for monitoring physiological conditions of a subject.

### BACKGROUND OF THE INVENTION

Sudden Infant Death Syndrome (SIDS) is a syndrome in which an infant dies suddenly in its sleep due to stopping breathing.

A variety of methods for monitoring a patient's vital signs are available for doctors and care-givers these days. These range from baby monitors, which transmit the sounds and even video of a baby to its parents in a different room, to pregnant women's contractions monitors, to EKG or ECG monitors in a hospital that monitor the vital signs of a patient.

Bar Hayim (WO 2009/083980) discloses a disposable monitor that includes an adhesive sensing unit that includes a flexible conductive wire wherein the sensing unit is attached to the skin of a person being monitored.

Rahamim (WO 2009/050702) discloses an apnea detector. It incorporates a capacitive type sensor that emits an alert signal upon detecting symptoms of apnea. In one embodiment, a detector unit is in communication with a curvature sensor adapted to detect a variable curvature of a subject body surface resulting from breathing patterns of a subject. The detector unit is attached to an article of clothing of the subject. A monitoring system comprises a detector unit for detecting one or more subject related parameters of interest and for emitting acoustical information after determining that a subject related parameter of interest has a predetermined status, and a stationary unit disposed within an audible range of the detector unit for receiving the emitted acoustical information.

Scanlon (U.S. Pat. No. 5,515,865) discloses an apparatus to monitor a living being's breathing movement and, in the absence of breathing movement, the apparatus attempts to stimulate breathing. The apparatus has a base member that may be a pad placed under the baby's body.

Young (U.S. Published Patent Application 2008/0077020) discloses a method and apparatus for monitoring vital signs remotely. It describes the use of a single sensor to monitor the cardiopulmonary activity of a patient using a ballistograph.

The current state in the field of monitoring a patient's vital signs involves large and cumbersome devices which can be very uncomfortable for the patient and may limit their use with babies and infants due to their size. The devices' size and complexity dictates the price of such devices to be high and that the devices must be reused and cannot be disposable.

Furthermore, most of today's devices include a sensor that is able to sense only one type of vital sign and, in case one needs to monitor several vital signs simultaneously, one is required to use several devices.

An important aspect in preventing SIDS and sleep apnea is said to do with the air composition in the vicinity of the baby. Elements such as air temperature, humidity and presence of poisonous gases such as carbon monoxide (CO) or cooking gases (such as Propane) may contribute to SIDS and sleep apnea.

There is therefore a need in the art for a system and method to incorporate one or more sensors into a cheap, single, small and disposable device, possibly manufactured into diaper and/or clothes. Another need is for a device that could control environmental influencing devices (air conditioning, electric fan, ionizer etc.) and also monitor the air in the vicinity of the baby as part of the prevention of SIDS or sleep apnea.

### SUMMARY OF THE INVENTION

According to some embodiments, there are provided unique methods, devices and systems, having a sensor for monitoring physiological conditions of a subject body. In particular, the methods, devices and systems may be used for monitoring the physiological condition of a baby. The methods, devices and system disclosed herein enable a cheap, non invasive, accurate, small and disposable means for providing real time monitoring of physiological condition of a subject being monitored. In particular, the methods, devices and systems disclosed herein provide means to monitor physiological conditions of a baby and to aid in prevention of Sudden Infant Death Syndrome (SIDS), sleep apnea as well as additional health related conditions, such as, for example, elevation of body temperature of the baby. Additionally, the devices, methods and system are able to control over environmental influencing devices (such as, for example, air conditioning, electric fan, ionizer, window openers, and the like) and are also able to monitor environmental parameters that are related to breathing in the vicinity of the subject (such as, for example, temperature, humidity, carbon monoxide (CO) concentration, light, and the like).

According to some embodiments, there is provided a sensor wherein application of pressure on the sensor causes changes in the resistance of the sensor, where the level of pressure is relative to the resistance. According to some embodiments, the sensor may be an elastic resistive sensor. In some embodiments, the sensor may be an electrically conductive sensor, such as, for example, conductive wires or printed circuit board (PCB) traces or solder pads in conjunction with conductive fabric, conductive paper, conductive paint, conductive ink, elastomer, piezoresistive sheet, or the like. In some embodiments, the elastic resistive sensor may be made of a foam material soaked with conductive electrolyte that may further be contained in a non-electrically-conductive housing, such as, for example, an air tight polymer cover.

According to some embodiments, there is provide a system for monitoring the physiological conditions of a subject body and to issue/provide an alert when these physiological conditions have changed to the detriment of the subject. In some embodiments, the physiological conditions are breath related parameters, such as, for example but not limited to: rate of breathing (number of breaths per minute), deepness of breath, lack of breathing, and the like and combinations thereof.

In some embodiments, the system may include a sensor of the physiological condition(s) of a subject body and which issues signals based on those physiological conditions. A receiver in proximity to a responsible overseer detects these signals and issues an appropriate warning signal/alert.

According to some embodiments, the sensor of the system may be a sensor (such as an elastic resistive sensor) wherein pressure on the sensor causes changes in resistance of the sensor, where the level of pressure is relative/proportional to the resistance. Additionally, a tactile element (such as, for example, a vibrator) may be included to alert/move the subject to reinvigorate him to restore normal physiological conditions. In some cases, there may also be a monitor of local environmental conditions.

According to some embodiments, the present invention may improve a sleeping baby's safety and prevents SIDS based on the monitoring of baby's physiological conditions, such as breathing, and analyzing them by a system and method that incorporate one or more sensors into a single, small and disposable device, possibly manufactured into the diaper during the diaper manufacturing or at a later time, or possibly, in the form of a sticker to be placed on a baby's diaper. In some embodiments, the system may further include a detector that is able to monitor/detect various breath related environmental conditions/parameters in the vicinity of the baby subject (such as, of example, temperature, light, humidity, CO concentration, and the like) and to further affect such conditions by controlling the operation of various devices (herein after also referred to as “electrical appliances”) that may influence the environmental parameters (such as, for example, air conditioning, electric fan, ionizer, electric window openers, and the like). In some embodiments, the system may be used to monitor the air/atmosphere in the vicinity of the subject baby as part of the prevention of SIDS or sleep apnea.

In some aspects, the present invention overcome deficiencies of the background art by providing a system and method that may include one or more of the following units: (a) a “subject unit” (also referred herein to as: “wearable subject unit” or “diaper unit”) placed within or on a garment used by the subject. For example, the subject unit may be placed on or within a baby's disposable diaper (optionally embedded within the diaper during manufacturing of the diaper); (b) a “crib unit” (“CU” in short, also named hereinafter as “platform unit”) for receiving/relaying and processing data. The platform unit may be placed in the near vicinity of the subject, and may be permanently or transiently attached to, for example, but not limited to:, a bed, a crib, a stroller, a room, high chair, car safety seat, and the like.; (c) an “overseer unit” (also named hereinafter as “supervisor unit” or “parents' unit”), which is a receiver and monitor unit located away from the subject's vicinity; (d) a “tactile unit” (for example, a vibrating unit); and (e) at least one “control unit” (also referred to as “interface unit”), such as, for example, electrical appliance control unit (with its corresponding electrical appliance).

According to some embodiments, the diaper unit may include one or more sensors (such as, for example, micro-sensors) to monitor the baby's breathing by analyzing the mechanical movement of the lower abdomen to detect various breath related events, such as, for example, cessation of breath, shortness of breath, breath rate, rhonchus and cessation of breath during sleep (sleep apnea). In some embodiments, the diaper unit also may include a body temperature sensor (such as a thermometer for measuring the baby's body temperature).

Exemplary sensors for use in the diaper unit are, for example, nut not limited to: an electrically conductive elastomer made by Chomerics, (part number: 10-04-2463-S6305), a conductive paper made by PASCO (part number: PK-9025), and Piezo resistive sheet (Velostat™). Additional options for movement sensors may include, for example, but not limited to: conductive fabrics such as those manufactured by Hebei Metals Inc. Exemplary fabrics that can be used include SS Mesh Shielding Fabric, ESD Static Fabric, Staticot™, EeonTex (Catalog number: LG SLPA 10E4, made by EEONYX LTD). Additionally or alternatively, a piezoelectric film may be used, such as, for example, piezoelectric film made by Meas, part number LDT0-028K/L w/crimps.

Further options for movement/pressure sensors may be, for example, a generally non-conductive foam/sponge material soaked in a conductive electrolyte.

According to some embodiments, the elastic resistive sensor may activated (automatically or manually), prior to use. In some embodiments, there is provided an activator for electrically activating the elastic resistive sensor, wherein the activator may be selected from the group consisting of: a strain gauge, a pull strip, a circuit-shortening conductive patch, a non-conductive pull strip on a battery contact, a reed switch, a battery comprising a built-in activation mechanism and a cover for activating a Zinc-air battery.

According to some embodiments, the electronic components of various units of the system may be packaged into a single device (Application Specific Integrated Circuit—ASIC) or may be composed using discrete components. This is true for the diaper unit as well as to any of the other units described hereinafter.

According to some embodiments, the platform unit may communicate with the diaper unit using various communication routes, such as, for example, acoustic communications in audible frequencies or ultrasonic frequencies, and optionally, using a wireless link, such as radio-frequency (RF) link.

In some embodiments, the data from the diaper unit's sensors is transmitted to the platform unit to be analyzed. Should the readings from the diaper unit exceed pre-set/predetermined limits; the platform unit may send an alert to the overseer unit via, for example, a wireless route. The overseer unit may, in turn, alert the overseers (such as for example, parents) by means of auditory and/or visual and/or tactile means (alarm, flashing lights, vibrations, etc.). When using an electromagnetic wireless link, it is possible to use common methods of low-range RF communications, such as Zigbee, Bluetooth and DECT.

In some embodiments, in order to prevent false alarms, the acoustic communications from the diaper unit may operate in a different frequency range than that of a baby's cry which is in the range of 1,000 Hertz (Hz) to 5,000 Hz, centered around 3,500 Hz, as well as from a baby's voice centered around 400 Hz.

According to some embodiments, the platform unit may be equipped with a digital signals processor (DSP) which can analyze the signals coming from the diaper unit and distinguish between an alert and other sounds such as a baby's cry, people speaking, etc.

In some embodiments, the acoustic communication from the diaper unit to the platform unit may be encoded such that only the corresponding platform unit would communicate with its diaper unit. The encoding can be done by specific timings and frequency of the sounds generated by the diaper unit.

In other embodiments, when an alert, which is indicative of breath related condition (such as, for example, apnea, breath rate below a predetermined threshold and the like) is activated, the platform unit may signal the tactile unit to start moving the crib (for example, by vibrating/rocking the crib). Research shows that vibrating a sleep apnea subject may re-initiate spontaneous breathing.

In some embodiments, in case that the air in the vicinity of the baby does not meet the pre-defined specifications/characteristics/limits, the system may further control external electrical appliances (devices) that can influence the baby's immediate vicinity. For example, in case the temperature is not within the pre-defined specification, the system may operate an air conditioner, an electrical fan an ionizer, and the like. For example, in case the CO concentration in the baby's vicinity is too high, the system may operate an electrical window opener to allow fresh air to come into the room. In some embodiments, once the issued alert had been addressed, the overseer may reset the system and halt the alarm(s).

According to some embodiments, the diaper unit may be in the form of a disposable sticker, that may be packaged in a sticker dispenser. The sticker dispenser may be designed in such a manner, that the diaper unit is powered up/activated only after it is taken out of the sticker dispenser. Furthermore, in some embodiments, it can also be powered by detecting initial vibrations after mounted on the abdomen, as explained hereinafter with respect to the disposable sensor.

According to some embodiments there is provided a system for monitoring breathing of a subject, the system comprising: (a) a wearable subject unit comprising: an elastic resistive sensor positionable such that breathing motion of the subject applies mechanical pressure to said elastic resistive sensor, wherein said elastic resistive sensor is configured to change its resistance responsive to the mechanical pressure, and a transmitter configured to wirelessly transmit a signal based on the change in resistance of the elastic resistive sensor; and (b) a platform unit comprising a receiver and being positionable in wireless transmission range with said subject unit, said platform unit configured to receive said signal from said subject unit and to issue an advisory signal indicative of the breathing of the subject.

According to some embodiments, the elastic resistive sensor comprises an elastic, electrically-conductive material contained within a non-electrically-conductive housing. The elastic, electrically-conductive material is selected from the group consisting of: foam soaked in a conductive electrolyte, a conductive fabric, a conductive paper, conductive paint, conductive ink, a conductive elastomer and a piezoresistive sheet.

In some embodiments, the wearable subject unit is embedded within a disposable diaper.

According to some embodiments, the transmitter of the wearable subject unit is a radio frequency (RF) transmitter, and the receiver of the platform unit is an RF receiver. In some embodiments, the transmitter is an acoustic transmitter, and said receiver of said platform unit is an acoustic receiver

According to some embodiments, the system may further include a remote unit configured to receive the advisory signal from the platform unit and to issue an alert to a supervisor of the subject.

According to some embodiments, the system may further include a tactile unit configured to reinvigorate the subject based on the signal from the wearable subject unit, when the signal indicates lack of breathing or breathing rate below a predetermined threshold.

According to some embodiments, the system may further include an environmental detector configured to detect at least one environmental parameter potentially affecting the breathing of the subject, the at least one environmental parameter may be selected from the group consisting of: temperature, humidity, light and carbon monoxide.

According to some embodiments, the system may further include an interface unit configured to affect the at least one environmental parameter in order to improve the breathing of the subject.

In some embodiments, the system may further include an activator for electrically activating the elastic resistive sensor, wherein the activator is selected from the group consisting of: a strain gauge, a pull strip, a circuit-shortening conductive patch, a non-conductive pull strip on a battery contact, a reed switch, a battery comprising a built-in activation mechanism and a cover for activating a Zinc-air battery.

According to some embodiments, there is provided a disposable diaper comprising: an elastic resistive sensor embedded within said disposable diaper and being positionable such that breathing motion of a subject wearing the disposable diaper applies mechanical pressure to said elastic resistive sensor, wherein said elastic resistive sensor is configured to change its resistance responsive to the mechanical pressure; and a transmitter embedded within said disposable diaper and being configured to wirelessly transmit a signal based on the change in resistance of the elastic resistive sensor.

In some embodiments, the elastic resistive sensor comprises an elastic, electrically-conductive material contained within a non-electrically-conductive housing. The elastic, electrically-conductive material may be selected from the group consisting of: foam soaked in a conductive electrolyte, a conductive fabric, a conductive paper, conductive paint, conductive ink, a conductive elastomer and a piezoresistive sheet.

In some embodiments, the transmitter may be a radio frequency (RF) transmitter. In some embodiments, the transmitter may be an acoustic transmitter.

According to some embodiments, there is provided a disposable diaper comprising an elastic resistive sensor embedded within said disposable diaper and being positionable such that breathing motion of a subject wearing the disposable diaper applies mechanical pressure to said elastic resistive sensor, wherein said elastic resistive sensor is configured to change its resistance responsive to the mechanical pressure; a transmitter embedded within said disposable diaper and being configured to wirelessly transmit a signal based on the change in resistance of the elastic resistive sensor; and an activator for electrically activating said elastic resistive sensor.

In some embodiments, the activator may be selected from the group consisting of: a strain gauge, a pull strip, a circuit-shortening conductive patch, a non-conductive pull strip on a battery contact, a reed switch, a battery comprising a built-in activation mechanism and a cover for activating a Zinc-air battery.

In further embodiments, the elastic resistive sensor comprises an elastic, electrically-conductive material contained within a non-electrically-conductive housing. The elastic, electrically-conductive material may be selected from the group consisting of: foam soaked in a conductive electrolyte, a conductive fabric, a conductive paper, conductive paint, conductive ink, a conductive elastomer and a piezoresistive sheet.

In further embodiments, the transmitter may be a radio frequency (RF) transmitter. In some embodiments, the transmitter may be an acoustic transmitter.

Other objects, features and advantages of the present invention will become apparent upon reading the following detailed description in conjunction with the drawings and the claims.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 is an isometric view of an illustrative, exemplary embodiment of a monitoring system for a baby, equipped with a diaper unit (DU), a platform unit (CU), and a vibrating unit (VU);

FIG. 2 is a schematic illustration of an exemplary embodiment of a monitoring and alerting system (MAS);

FIG. 3 is a block diagram of the monitoring and alerting system (MAS);

FIG. 4a is a front, perspective view of an illustrative, exemplary embodiment of a diaper equipped with a diaper unit;

FIG. 4b is an isometric view of an illustrative, exemplary embodiment of a diaper unit;

FIG. 4c is an isometric view of an illustrative, exemplary embodiment of a diaper unit containing conductive fabric sensors;

FIG. 4d is an isometric view of an illustrative, exemplary embodiment of a diaper unit containing a sponge material sensor with contacts for resistive sensing;

FIG. 4e is a block diagram of an exemplary embodiment of the diaper unit;

FIG. 4f is an isometric top view of an illustrative, exemplary embodiment of a diaper unit containing a piezoelectric film sensor;

FIG. 4g is an isometric bottom view of the diaper unit containing a piezoelectric film sensor shown in FIG. 4f;

FIG. 4h is an isometric top view of an illustrative, exemplary embodiment of a diaper unit containing a resistive sensor;

FIG. 4i is an isometric bottom view of the diaper unit containing a resistive sensor shown in FIG. 4h;

FIG. 4k is an isometric view of an illustrative, exemplary embodiment of a diaper unit containing multi sensor;

FIG. 5a is an isometric view, schematic illustration of an illustrative, exemplary embodiment of a platform unit (CU), according to embodiments of the present invention;

FIG. 5b is a schematic block diagram of the platform unit, according to embodiments of the present invention;

FIG. 6a is an isometric view of an illustrative, exemplary embodiment of a parents' unit (PU);

FIG. 6b is a block diagram of the parents' unit;

FIG. 7a is an isometric view of an illustrative, exemplary embodiment of a sticker dispenser (SD);

FIG. 7b is a block diagram of the sticker dispenser;

FIG. 8a is an isometric view of an illustrative, exemplary embodiment of a vibrating unit (VU);

FIG. 8b is a block diagram of the vibrating unit (VU);

FIG. 9 is a block diagram of an electrical appliance control unit (EACU);

FIG. 10 is a flow chart that illustrates a method for monitoring a baby's physiological conditions, for alerting people to conditions, and for operating electrical appliances;

FIG. 11a is an isometric view of an alternate embodiment of a diaper unit (DU);

FIG. 11b is an isometric view of another alternate embodiment of a diaper unit (DU);

FIG. 11c is an isometric view of another alternate embodiment of a disposable diaper unit (DU);

FIG. 12 is a view of a diaper, partially broken away, with an embedded DU resistive sensor;

FIGS. 13a and 13b are graphs of experimental results of a DU resistive sensor;

FIG. 14a is a schematic illustration of an experiment setup, measuring various properties of a DU resistive sensor;

FIGS. 14b through 14e are graphs of experimental results of a DU resistive sensor;

FIG. 15 is a view, partially broken away, of a diaper equipped with a diaper unit with partial cutouts, and a first type of activation mechanism;

FIG. 16a is a sectional front view of a diaper equipped with a diaper unit, and a second type of activation mechanism;

FIG. 16b is a bottom view of a diaper equipped with a diaper unit, and a second type of activation mechanism;

FIG. 17a is an isometric view of an illustrative, diaper unit equipped with a pull strip assembly, as a third type of activation mechanism;

FIG. 17b is a top view of an illustrative, diaper unit equipped with a pull strip assembly, as a third type of activation mechanism;

FIG. 17c is a cross sectional view along line a-a of FIG. 17b;

FIG. 18 is a schematic illustration of a diaper equipped with a diaper unit, and a fourth type of activation mechanism;

FIG. 19a is an isometric view of an illustrative diaper unit and another activation mechanism;

FIG. 19b is a side view of the device shown in FIG. 19a;

FIG. 20 is an isometric view of an illustrative diaper unit, connected to an array of DU resistive sensors laid out in a grid onto a resistive sensor support sheet using a set of conductive leads;

FIG. 21 is an isometric view of an illustrative diaper unit and another activation mechanism;

FIG. 22 is a view similar to FIG. 15, but showing another position for the activation mechanism;

FIG. 23 is a view similar to FIGS. 15 and 22, but showing another position for the activation mechanism;

FIG. 24 is an isometric view of an illustrative diaper unit and another discharge mechanism;

FIG. 25 is an isometric view of an illustrative diaper unit and another activation mechanism;

FIG. 26 is an isometric view of an illustrative, exemplary embodiment of a diaper unit containing a multi sensor;

FIG. 27 is an isometric view of an illustrative diaper unit similar to FIG. 4d and showing that the resistive sensor is printed with resistive ink or dye onto the diaper or embedded within the diaper;

FIG. 28 is an isometric view of a bent sensor;

FIG. 29 is an isometric view of an illustrative diaper unit and another activation mechanism;

FIG. 30 is an isometric view of an illustrative diaper unit and another discharge mechanism; and

FIG. 31 is an isometric view of an illustrative diaper unit and another activation mechanism.

### DETAILED DESCRIPTION

According to some embodiments, there are provided devices systems and methods for monitoring a subject's physiological conditions and for alerting changes in said physiological condition. In some embodiments, there are provided methods, devices and systems for monitoring physiological conditions of a baby, by non-invasive means, and for alerting changes in said conditions so as to prevent health related conditions, such as, for example, sleep apnea and SIDS.

As referred to herein, the term “subject” may refer, for example, to a baby, a child or an adult. For simplicity of presentation, the term “baby” is sometimes used to represent all types of subjects.

Referring now to the drawings, any internal elements, placed within the units' cases, are marked using a dashed line. The arrangement and shape of the elements as they appear in the drawings is exemplary only.

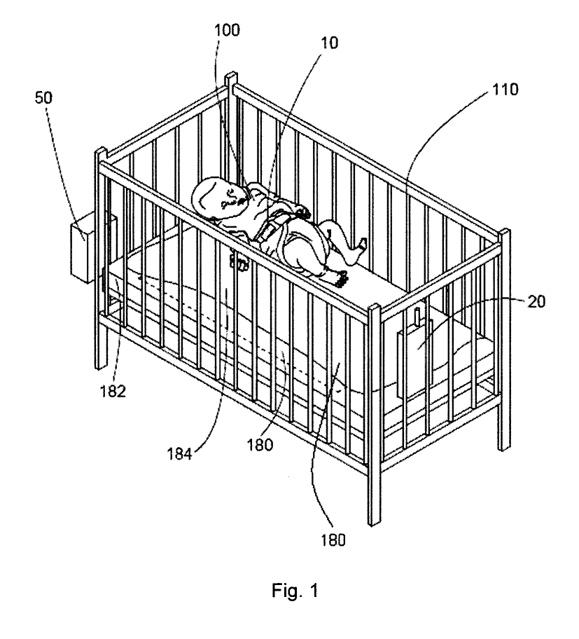

FIG. 1 is a view of a baby 100, equipped with diaper unit (DU) 10, laying in a crib 110 equipped with platform unit (CU) 20, and a vibrating unit (VU) 50, according to embodiments of the present invention.

The crib includes a mattress 182 that may include an embedded resistive sensor support sheet 180 or, alternatively, the mattress 182 may be covered with a bed sheet 184 that includes the resistive sensor support sheet 180. This sensor support sheet acts as a movement sensor.

The CU 20 is placed near the baby, possibly on the crib 110 itself, or on a nearby shelf or piece of furniture, and the VU 50 is fastened onto the crib 110 in order to ensure proper vibration of the crib 110.

It may be appreciated that the crib is shown for illustrative purposes only. Additional environments in which the baby is situated/located, such as, for example, a baby carriage, bassinet, or any other object in which a baby is resting or positioned may be used in the various embodiments.

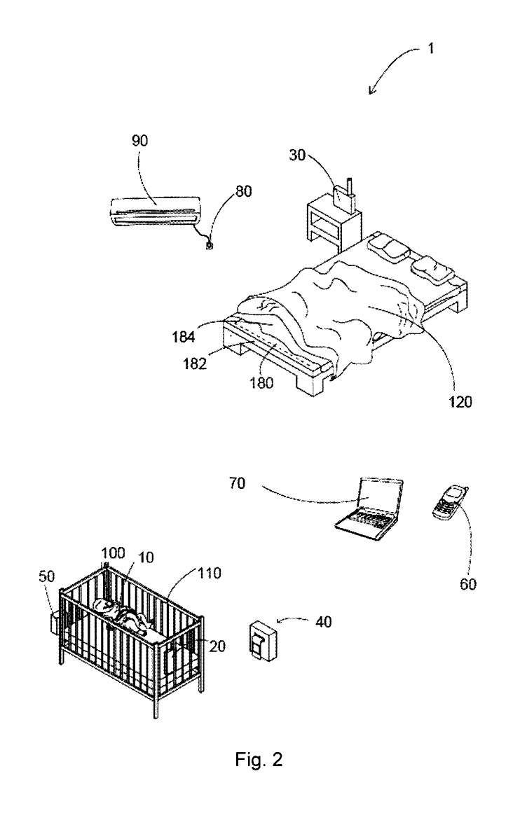

FIG. 2 shows a monitoring and alerting system (MAS) 1, according to some embodiments. The crib 110 together with the baby 100, DU 10, CU 20, VU 50 and optional sticker dispenser (SD) 40 may be placed away from the overseer (such as a parent) bed 120 and PU 30, which could even be in a different room. The cellular phone 60 and personal computer 70 may usually be away from the crib 110, as well.

Electrical appliance control units (EACU) 80 with their corresponding electrical appliances 90 are in the vicinity of the crib 110 and baby 100 in order to increase their effectiveness. (For illustrative purposes only one electrical appliance control unit with its corresponding electrical appliance is shown, but any number of such devices may be used, as necessary to control the environment.)

When the person being monitored is older than a baby and uses a bed, rather the person's bed can include a mattress 182 that may include an embedded resistive sensor support sheet 180 or may be covered with a bed sheet 184 that includes a resistive sensor support sheet 180 in order to act as a movement sensor.

FIG. 3 is a block diagram of the monitoring and alerting system (MAS) 1, according to embodiments of the present invention, including the major units of the MAS 1 and their inter-connections. Data gathered by the sensors of the DU 10 is transmitted by the DU 10 as a diaper unit to platform unit communication 91 to the platform unit (CU) 20 and is then transmitted, as necessary, as a SMS message 92 from the CU 20 to a telephone (shown as cellular phone 60), as an e-mail message 93 to personal computer 70, as platform unit to parents' unit RF communication 94 to the parents' unit (PU) 30, as platform unit to electrical appliance control units RF communication 95 to the electrical appliance control units 80, and/or as platform unit to vibrating unit RF communication 96 to the vibrating unit 50. The electrical appliance control units (EACU) 80 control electrical appliances 90. In some embodiments, the personal computer 70 may be equipped with an application for monitoring the system.

The sticker dispenser 40 generally may or may not communicate with other components/units of the system. In some embodiments, the sticker dispensed may communicate by any communication route, such as, for example, by wireless means.

The MAS 1 may also include a diaper unit activation mechanism 12s (not shown in FIG. 3 but herein after described), which is operatively connected to the diaper unit 10. The diaper unit activation mechanism 12s can be made so as not to require any special action for the purpose of activation of the diaper unit 10, namely the diaper unit 10 can be activated by a simple action such as the opening of the diaper.

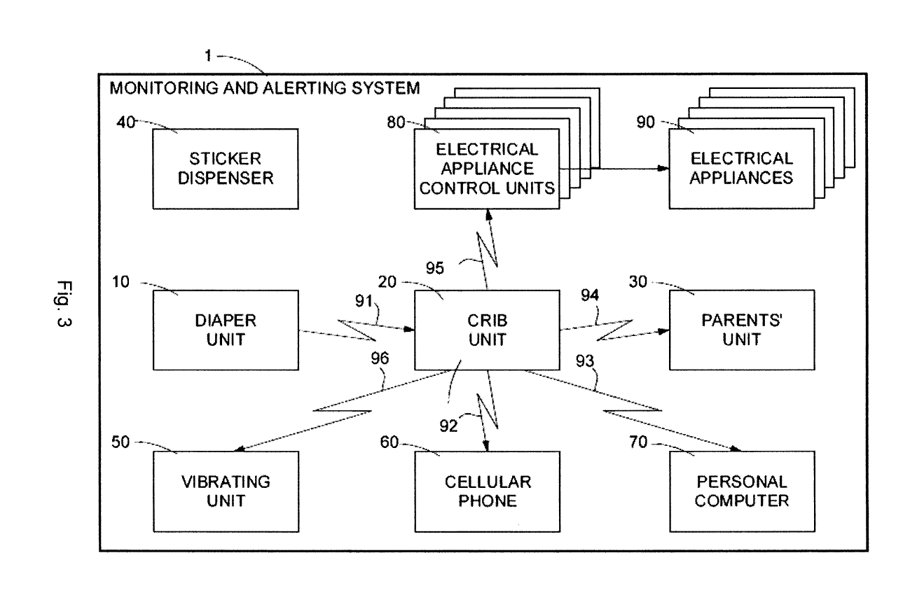

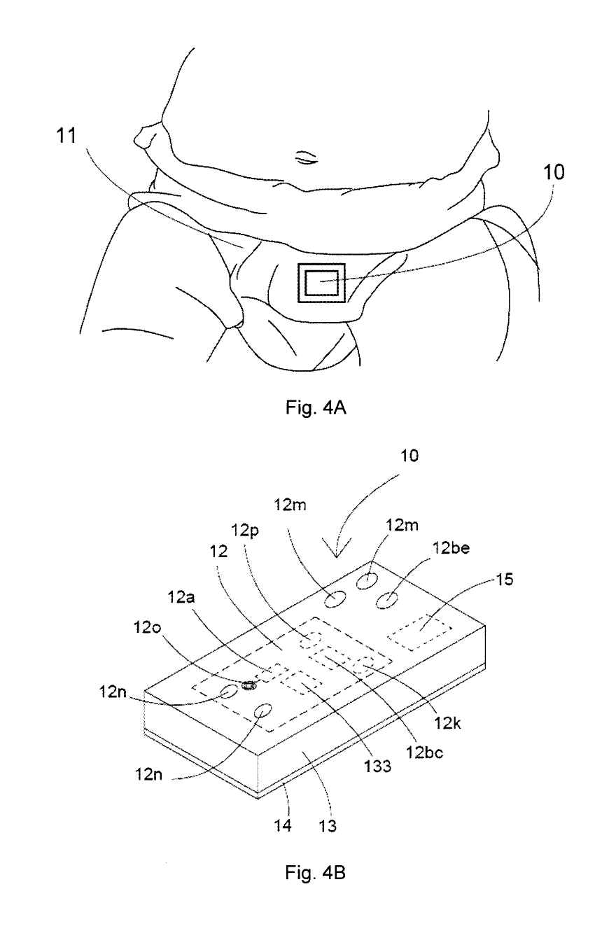

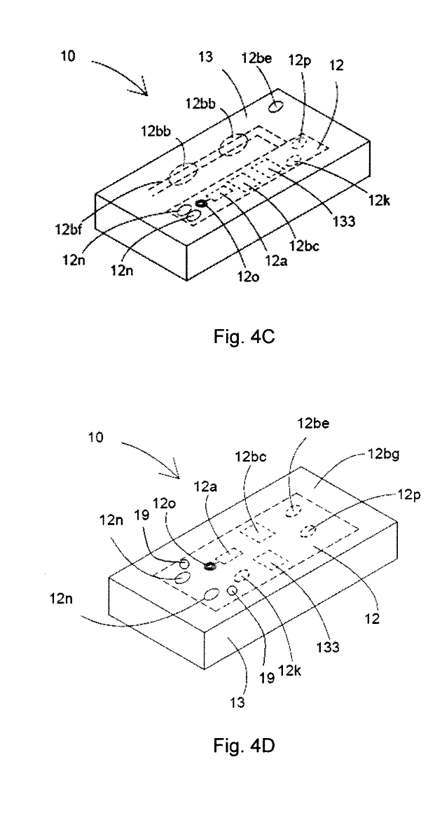

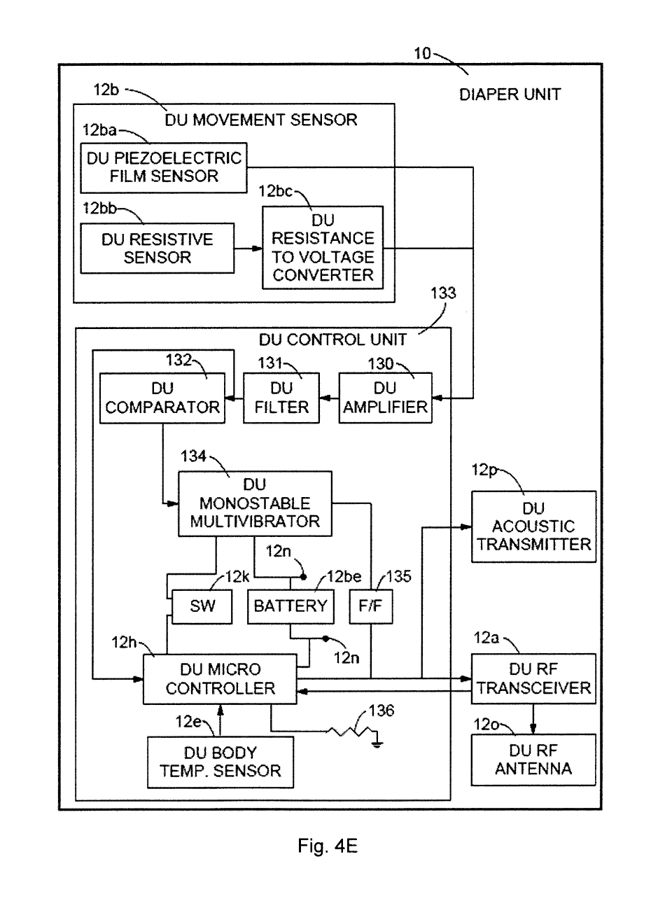

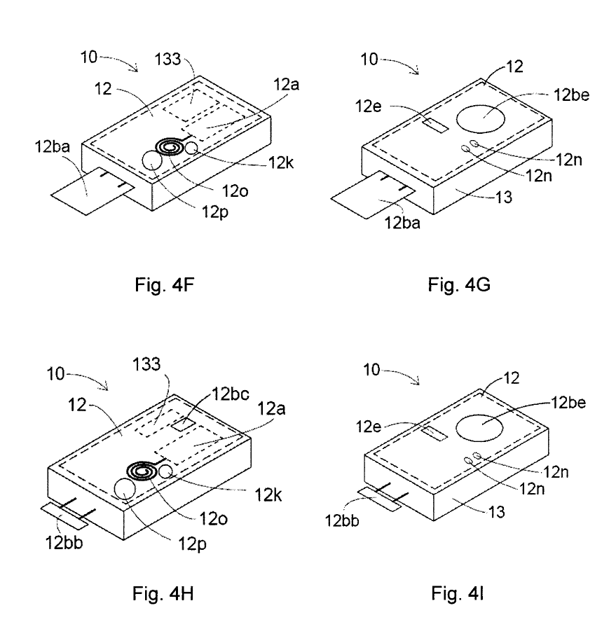

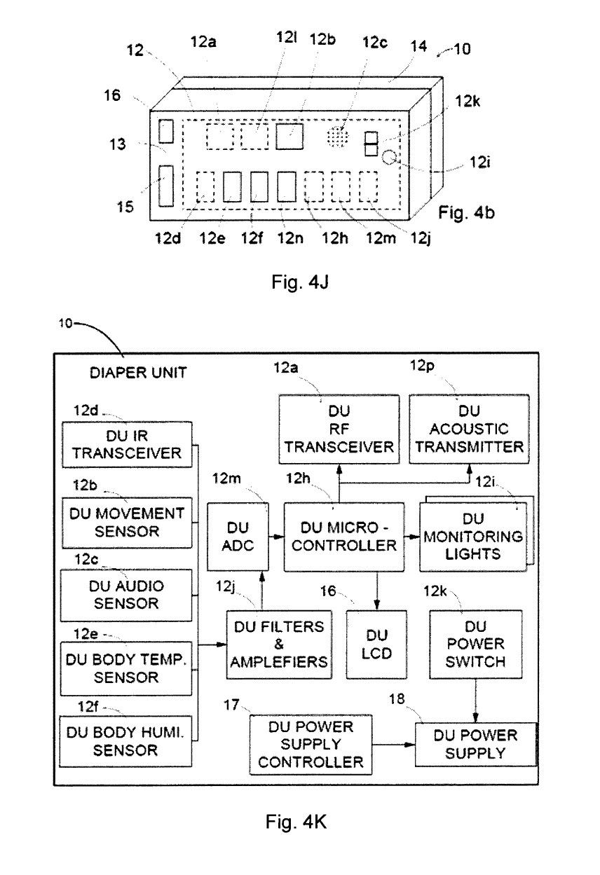

FIG. 4a is a perspective view of a diaper 11 equipped with diaper unit 10, according to embodiments of the present invention. The DU 10 can be placed on the diaper 11 or inside the diaper 11.

The DU 10 can be packaged in a sticker-like package by various means, such as, illustrated, for example, by any of the embodiments described in following figures.

The functionality of each of these elements is as described by name, and will be detailed in the description of the following illustrations. The geometrical shapes of the elements as shown in the present illustration, as well as their relative size and locations relative to each other, are in no way limiting the present invention.

FIG. 4b is an isometric view of a DU 10 in a sticker configuration, according to embodiments of the present invention. In the present illustration, the DU 10 is equipped with a DU adhesive tape 14 and is encased in a sensing module sticker case with the DU printed circuit board 12 and DU magnetic flux direction detector device 15 disposed inside it. All of the components are enclosed within a DU sensing module case 13.

FIG. 4c is an isometric view of a DU 10 containing conductive fabric sensors, according to embodiments of the present invention. The DU resistive sensors 12bb in this figure are made of a conductive fabric material and are connected to the DU printed circuit board 12 using a pair (or more) of DU conductive wires 12bf, possibly yarn or nonwoven wires or printed circuit board traces or solder pads. The pressure applied on the fabric, causes changes in the resistance of the wires as the fabric touches the wires (traces or solder pads), where the pressure is relative to the resistance. By way of comparison and explanation and without being limited to any theory or mechanism, it would be the same as, if a resistor's components are connected in parallel or series in an electric circuit, any change of the resistance value or the addition or removal of a resistor will change the circuit resistance as a whole. The resistance changing feed into the converter can be a discrete chip or made integral in the microcontroller. This converter is composed of a voltage divider and an operational amplifier (hereinafter, OpAmp). The change in the resistance causes a change in the voltage divider output, thereby resulting in a change in the current fed into the OpAmp. Then, the OpAmp output is changing within the limits of its supply voltage. In some embodiments, the limits are between about 0 volts to about 3 volts. Therefore, the voltage may swing in this range at the output of the OpAmp.

Alternatively, in some embodiments, the fabric sensor can be implemented on the flexible or thin rigid PCB (printed circuit board) which would also contain the electronics. In this embodiment, it is also possible to discharge the DU battery 12 by using a pair of DU discharge contacts 12n.

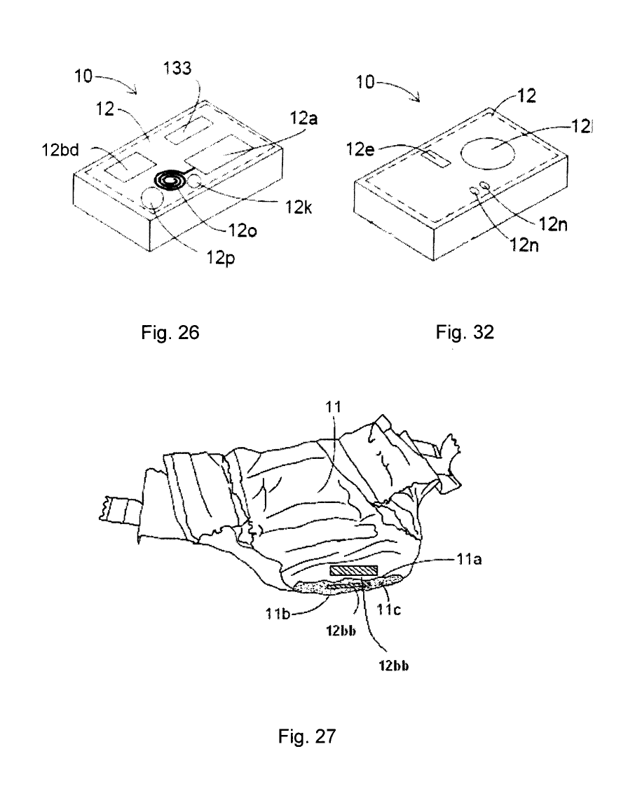

As shown in FIGS. 26 and 32, the elements shown as components of the DU in the present illustration comprise one possible configuration out of various options, and include: a DU printed circuit board 12, a DU RF transceiver 12a, a DU movement sensor 12b, a DU audio sensor 12c, a DU infra-red transceiver 12d, a DU body temperature sensor 12e, a DU body humidity sensor 12f, a DU microcontroller 12h, DU control lights 12i, DU filter and amplifiers 12j, a DU power switch 12k, DU filters and amplifiers 12l, a DU analog to digital converter (ADC) 12m, a DU sensing module case 13, DU adhesive tape 14, a DU magnet 15, and a DU liquid crystal display (LCD) 16.

FIG. 4K is a schematic block diagram of an exemplary embodiment of diaper unit 10, according to embodiments of the present invention. The diaper unit 10 includes several sensors such as the DU movement sensor 12b, the DU audio sensor 12c, DU infra-red transceiver 12d, DU body temperature sensor 12e and the DU body humidity sensor 12f which continuously send their readings to the DU filter and amplifiers 12j. The DU filters and amplifiers 12j send the filtered and amplified sensor readings to the DU analog to digital converter (ADC) 12m for digitization. The digitized data is then sent to the DU microcontroller 12h for analysis. The DU microcontroller 12h analyzes the received data and outputs the corresponding information on the DU monitoring lights 12i and the DU liquid crystal display (LCD) 16. If required, the DU microcontroller 12h sends out a diaper unit to platform unit communication 91 to the platform unit 20 using the DU radio frequency transceiver 12a or the DU acoustic transmitter 12p.

In order for the DU 10 to function properly, it includes a DU power supply 18 which is controlled by the DU power supply controller 17 for voltage stability, current drain and power consumption monitoring and can be turned on and off by using the DU power switch 12k.

FIG. 4d is a DU 10 containing a sponge/foam material sensor, according to embodiments of the present invention. The sponge/foam material is usually inert, but when it is soaked with highly conductive multi-purpose electrolyte (such as “Signa Spray” made by Parker) and packaged in an air tight polymer cover, it changes its resistivity when pressure is applied to it, and it is also possible in the foam manufacturing process to mix the foam resin with any conductive material such as, copper, silver, etc.

In this configuration, the DU printed circuit board 12 and all of the components on it may be embedded within the sponge/foam material.

In this embodiment it is also possible to discharge the DU battery 12be using a pair of DU discharge contacts 12n.

FIG. 4e is a diaper unit 10, according to embodiments of the present invention. This exemplary embodiment of the diaper unit 10 contains a DU movement sensor 12b such as a DU piezoelectric film sensor 12ba and/or a DU resistive sensor 12bb followed by a DU resistance to voltage converter 12bc, or any other suitable device for measuring movement.

The DU resistive sensor 12bb may be an elastomer based sensor, a fabric based sensor, a paper based sensor, a piezoresistive sensor, a sponge based sensor foam based sensor or any other material that changes an electrical property as it deforms when a pressure is applied to it.

An elastomer is a material that changes its resistivity when it is stretched or bent. A conductive fabric sensor (such as made by EEONYX and called EeonTex, catalog number: LG SLPA 10E4), or conductive paper (such as PK-9025 made by PASCO), or Piezo resistive sheet (such as Velostat™) with conjunction with conductive yarn (such as “Silver plated Nylon Yarn” P/N PY16125 made by Statex Production & Distribution PLC) or with any other conductive wires/pcb solder pads is a combination that causes changes in the resistance when pressure is applied to them or by bending them. A sponge/foam based sensor uses a sponge/foam soaked with a highly conductive multi-purpose electrolyte (such as “Signa Spray” made by Parker) and packaged in an air tight polymer cover, and changes its resistivity when pressure is applied to it.

The output of the DU movement sensor 12b (which is a low voltage signal) is input to a DU control unit 133 and in particular to a DU amplifier 130 that amplifies the low voltage signal from the DU movement sensor 12b and the output of the DU amplifier 130 is input to a DU filter 131 for noise removal. The output of the DU filter 131 is input to the DU comparator 132. When the voltage level of the DU filter 131 output is high enough, above a predefined level, the DU comparator 132 outputs a logic “1” signal. Otherwise, the DU comparator 132 outputs a logic “0” signal. This comparison can also be done within the DU microcontroller 12h using an internal comparator, or sampling the DU filter 131 output by its (uC) internal ADC by averaging the digital counting, etc. The DU comparator 132 output is input to a single DU mono-stable multi-vibrator 134 with output logic level latch, implemented by a DU Flip-Flip 135, or by a DU microcontroller 12h with zero, one or more sensors such as a DU body temperature sensor 12e. Other sensors may also be added for additional functionality. The DU control unit 133 controls the DU RF transceiver 12a and/or the DU acoustic transmitter 12p, which communicates with the platform unit 20 (not shown in the present drawing). Depending on its inputs via the DU RF antenna 12o, the DU RF transceiver 12a also sends incoming communications from the platform unit 20 (not shown in the present drawing), to the DU control unit 133. These communications may contain system status updates. Power is supplied to the DU 10 components from the DU battery 12be and is activated by the DU power switch 12k.

In some embodiments, the mechanical pressure on the sensor may be different each time a diaper is put on the baby; therefore, the analog input voltage that the sensor will produce depends on its bending and is different on each occasion. The sensor produces resistance change and then DU resistance to voltage converter 12bc converts it to voltage for processing. This architecture may cascade with several comparators (block 132) and mono-stable multivibrators (block 134). Each of the comparators may be configured for different presets of input voltage, and also it might have a logic AND/OR/NOT/XOR gate (any combination) at the outputs of the mono-stable multivibrators in order to get the absolute decision for alerting without false alarms.

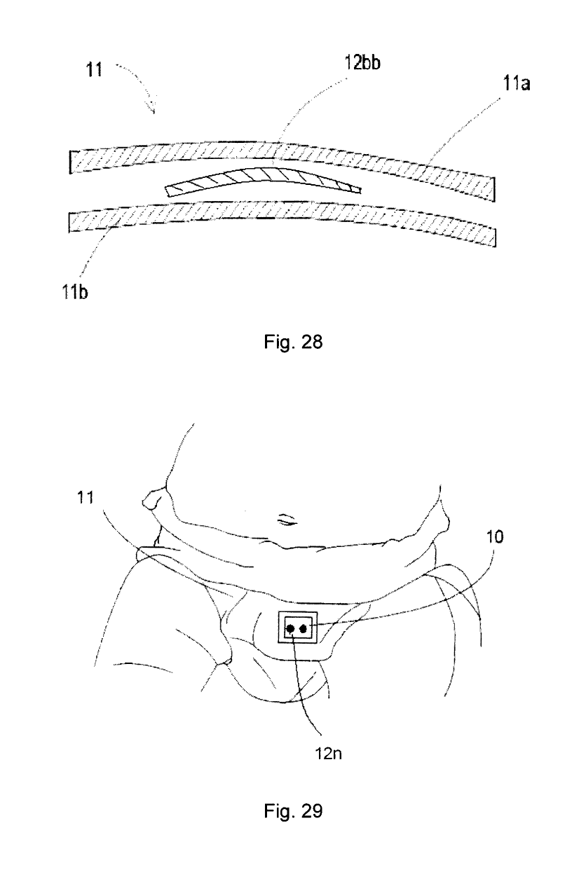

It is not possible to control the precise place and the tightness of the diaper when diapering a baby. Therefore in one embodiment (FIG. 28), a bent shape of the sensor may be used as a default. This may override the gap between the abdomen and the diaper.

When the two DU discharge contacts 12n are shorted, or when for a known/predetermined period of time there is no abdomen movement, the DU 12h may drive current through the DU load resistor 136, or any kind of load component or electronic circuit, in order to deplete the charge of the DU battery 12be.

FIG. 4f is a diaper unit 10 containing a piezoelectric film sensor 12ba, according to embodiments of the present invention. The elements shown as components of the DU 10 in FIG. 4f comprise one possible configuration out of various options of the top side of the DU 10, and include: a DU printed circuit board 12, a DU RF transceiver 12a, a DU acoustic transmitter 12p, a DU power switch 12k, a DU control unit 133, a DU piezoelectric film sensor 12ba and a DU RF antenna 12o.

FIG. 4g is a diaper unit 10 containing a piezoelectric film sensor 12ba, according to embodiments of the present invention. The elements shown as components of the DU 10 in FIG. 4g comprise one possible configuration out of various options of the bottom side of the DU 10, and include: a DU printed circuit board 12, a DU body temperature sensor 12e and a DU battery 12be.

FIG. 4h is a diaper unit 10 containing a resistive sensor, according to embodiments of the present invention. The elements shown as components of the DU 10 in FIG. 4h comprise one possible configuration out of various options of the top side of the DU 10, and include: a DU printed circuit board 12, a DU RF transceiver 12a, a DU acoustic transmitter 12p, a DU power switch 12k, a DU control unit 133, a DU resistive sensor 12bb, a DU resistance to voltage converter 12bc and a DU RF antenna 12o.

FIG. 4i is a diaper unit 10 containing a resistive sensor, according to embodiments of the present invention. The elements shown as components of the DU 10 in FIG. 4i comprise one possible configuration out of various options of the bottom side of the DU 10, and include: a DU printed circuit board 12, a DU body temperature sensor 12e, and a DU battery 12be.

With the configuration shown in FIG. 4d, the DU resistive sensor 12bb (FIG. 27) is printed with resistive ink or dye onto the diaper 11 or embedded within the diaper 11, and the DU 10 is attached onto the diaper so that the DU resistive sensor 12bb connects to the DU 10 by means of the two DU resistive sensor contacts 19.

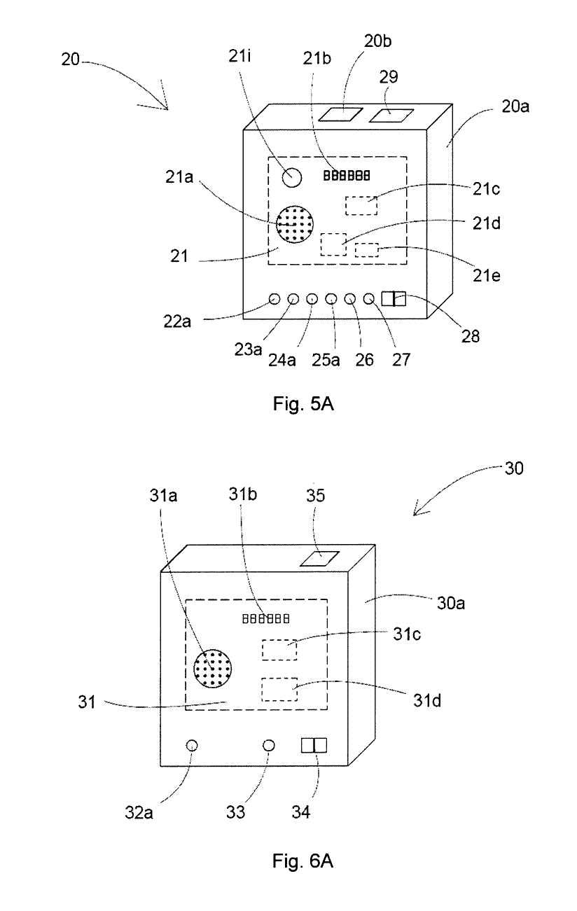

FIG. 5a is a platform unit (CU) 20, according to embodiments of the present invention. Some of the elements of the CU 20 are within the interior of CU 20 and are marked with a dashed line. The elements shown as components of the CU 20 in the present illustration comprise one possible configuration out of various options, and include: a CU case 20a, a CU magnet 20b, a CU printed circuit board 21, a CU loudspeaker 21a, a CU liquid crystal display 21b, a CU microprocessor 21c, a CU RF transceiver 21d, a CU cellular modem 21e, CU microphone 21i, a CU air temperature sensor 22a, a CU cooking gas sensor 23a, a CU carbon monoxide (CO) sensor 24a, a CU air humidity sensor 25a, CU control light 26, a CU power supply control light 27, a CU power switch 28, and a CU disable alarm button 29. The functionality of each of these elements is as described by name, and will be detailed in the description of the following illustration. The geometrical shapes of the elements as shown in the present illustration, as well as their relative size and locations relative to each other, are in no way limiting the present invention.

FIG. 5b is a block diagram of the platform unit 20, according to embodiments of the present invention. The platform unit 20 includes several sensors such as the CU cooking gas sensor 23a, the CU carbon monoxide sensor 24a, the CU air humidity 25a and the CU air temperature sensor 22a. Each sensor transmits its readings to the corresponding sensor interface unit (a CU cooking gas sensor interface unit 23b, a CU carbon monoxide (CO) sensor interface unit 24b, a CU air humidity sensor interface unit 25b and a CU air temperature sensor interface (IF) unit 22b respectively) which convert the sensor data into microcontroller readable data.

The CU microprocessor 21c analyzes the data from the sensors and outputs the appropriate signals to the CU liquid crystal display 21b (via the CU liquid crystal display controller 21f) and to the CU loudspeaker 21a (via the CU audio amplifier 21g). Additionally, the CU microprocessor 21c communicates with the CU RF transceiver 21d and the CU cellular modem 21e.

The CU microphone 21i listens for acoustic transmissions from the diaper unit 10 (shown in FIG. 4b) and sends its data to the CU digital signals processor (DSP) 21h to analyze and detect specific frequencies sent by the diaper unit 10. When the CU DSP 21h detects a valid communication, it signals the CU microprocessor 21c.

In some embodiments, when the DU is working, it may produce an acoustic sound in a known interval, which indicates a proper functionality when received by the CU.

The CU disable alarm button 29 signals the CU microprocessor 21c to turn off the alarm once it had started.

In order for the CU 20 to function correctly, it includes a CU power supply 28c which is turned on by the CU power switch 28a and is controlled and monitored by the CU power supply controller 28b. The CU power supply 28c has several CU power supply control lights 28d to display its status.

FIG. 6a is a parents' unit (PU) 30, according to embodiments of the present invention. Some of the elements of the PU 30 are within the interior of PU 30 and are marked with a dashed line. The elements shown as components of the PU 30 in the present illustration comprise one possible configuration out of various options, and include: a PU case 30a, a PU printed circuit board 31, a PU loudspeaker 31a, a PU liquid crystal display 31b, a PU microprocessor 31c, a PU RF receiver 31d, a PU cooking gas sensor 32a, a PU power supply control light 33, a PU power switch 34, and a PU disable alarm button 35.

FIG. 6b is a block diagram of the parents' unit 30, according to embodiments of the present invention. The parents' unit 30 includes several sensors such as the PU cooking gas sensor 32a, the PU carbon monoxide sensor 38a, the PU air humidity sensor 36a and the PU air temperature sensor 37a. Each sensor transmits its readings to the corresponding sensor interface unit (a PU cooking gas sensor interface (IF) unit 32b, a PU carbon monoxide sensor interface unit 38b, a PU air humidity sensor interface unit 36b and a PU air temperature sensor interface unit 37b respectively), which converts the sensor data into microcontroller readable data.

In some embodiments, the PU microcontroller 31c analyzes the data from the sensors and outputs the appropriate signals to the PU liquid crystal display 31b (via the PU liquid crystal display controller 310 and to the PU loudspeaker 31a (via the PU audio amplifier 31g). Additionally, the PU microcontroller 31c communicates with the PU RF receiver 31d.

The PU disable alarm button 35 signals the PU microcontroller 31c to turn off the alarm once it starts.

In order for the PU 30 to function correctly, it includes a PU power supply 33c which is turned on or off by the PU power switch 33a and is controlled and monitored by the PU power supply controller 33b. The PU power supply 33c has several PU power supply control lights 33d to display its status.

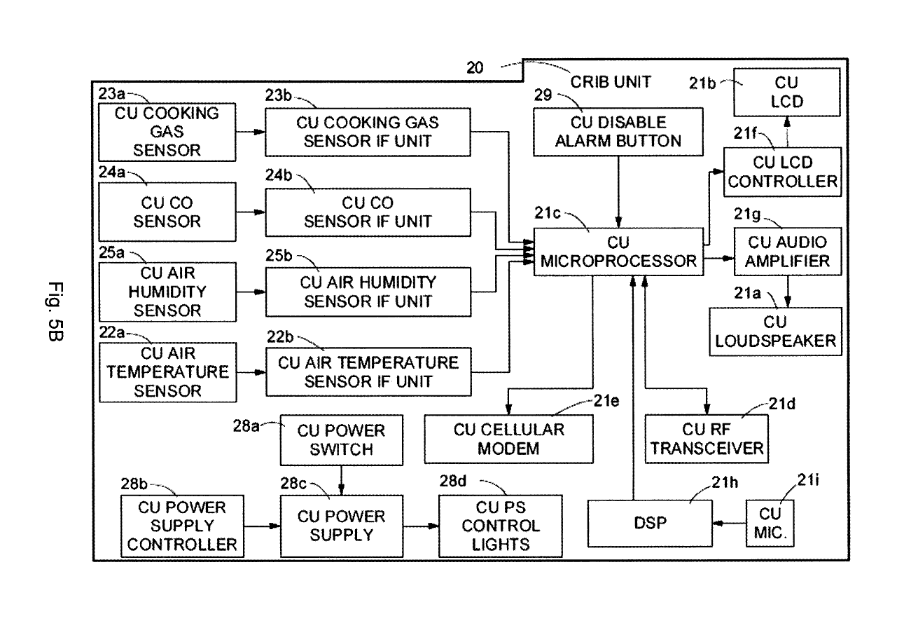

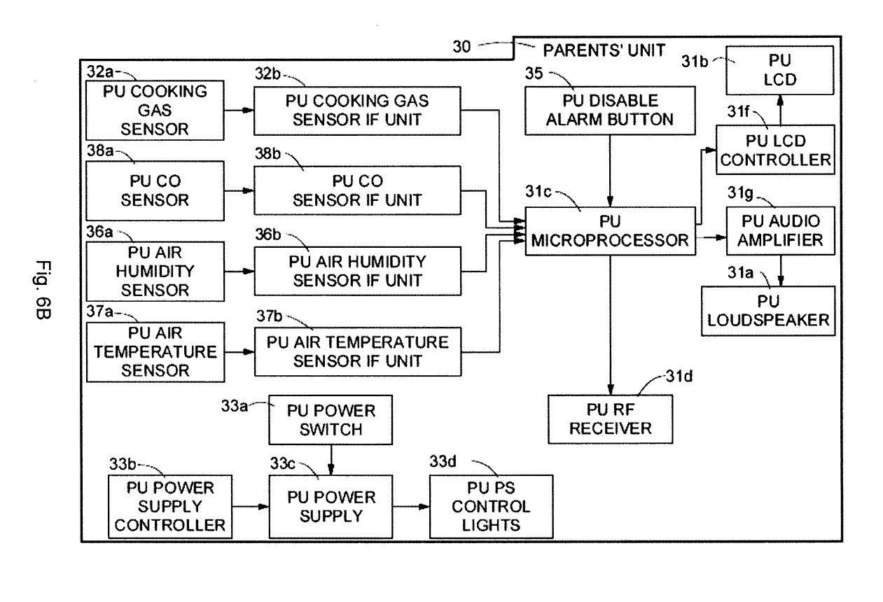

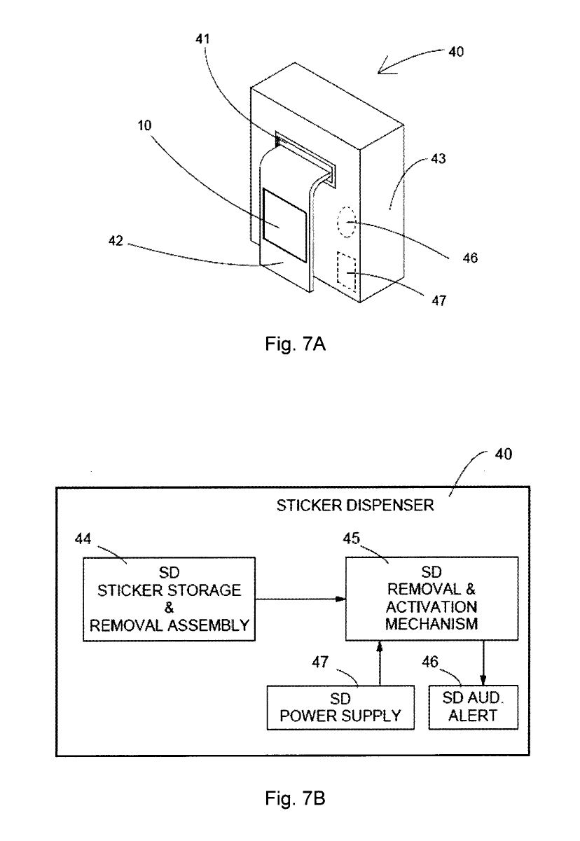

FIG. 7a is a sticker dispenser (SD) 40, according to embodiments of the present invention. The elements shown as components of the SD 40 in the present illustration comprise one possible configuration out of various options and include: Several diaper units (DU) 10 on a SD sticker strip 42, a sticker dispenser (SD) 40, a SD strip opening 41, a SD case 43, a SD auditory alert 46 and a SD power supply 47.

The sticker dispenser 40 holds the SD sticker strip 42 in the SD sticker and removal assembly. The DU 10 units are attached on the SD sticker strip 42, in a manner that enables their easy removal, for the purpose of using them as necessary. The SD sticker strip 42 comes out of the sticker dispenser 40 through the SD strip opening 41.

The geometrical shapes of the elements as shown in the present illustration, as well as their relative size and locations relative to each other, are in no way limiting.

FIG. 7b is a block diagram of the sticker dispenser 40, according to embodiments of the present invention. The individual DU 10 units (not shown in the present drawing) are stored on the sticker strip 42 (not shown in the present drawing) within the SD case 43 (not shown in the present drawing) inside the sticker storage a removal assembly 44. Once a DU 10 (not shown in the present drawing) is removed from the SD sticker strip 42 (not shown in the present drawing), the SD removal and activation mechanism 45 includes one polar magnet and the DU 10 is turned on automatically as its magnetic flux device (such as the Murata P/N:AS-M15TA-R) feels the fixed magnet within the SD removal and activation mechanism 45, and the DU 10 makes a specific sound to indicate it is active.

The SD auditory alert 46 will sound to indicate that the entire available DU 10 units in the SD 40 have been used up. In order to function properly, the SD 40 is equipped with a SD power supply 47.

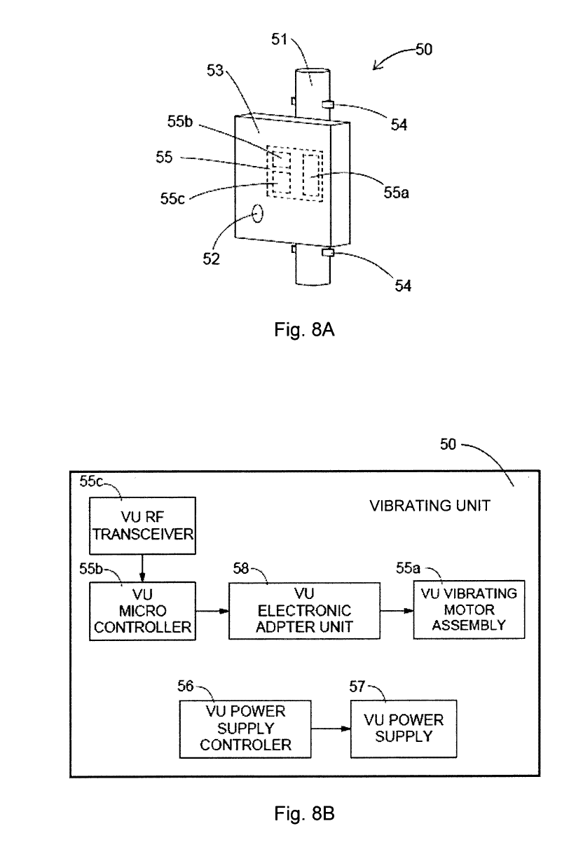

FIG. 8a is a vibrating unit (VU) 50, according to embodiments of the present invention. The elements shown as components of VU 50 in the present illustration comprise one possible configuration out of various options and include: a VU grip pole 51, a VU 18, power supply light 52, a VU case 53, a VU lock screw 54, a VU printed circuit board 55, a VU trembling motor assembly 55a, a VU microcontroller 55b, and a VU RF transceiver 55c.

The functionality of each of these elements is as described by name, and will be detailed in the description of the following illustration.

Some of the elements of the VU 50 are within the interior of VU 50 and are marked with a dashed line.

The geometrical shapes of the elements as shown in the present illustration, as well as their relative size and locations relative to each other, are in no way limiting the present invention.

FIG. 8b is a block diagram of the vibrating unit (VU) 50, according to embodiments of the present invention. The VU RF transceiver 55c is constantly waiting to receive the alert signal via the RF communication platform unit to vibrating unit RF communication 96 (not shown in FIG. 8b, shown in FIG. 3). Upon receiving the alert signal it signals the VU microcontroller 55b. The VU microcontroller 55b operates the VU electronic adapter unit 58, which in turn operates the VU trembling motor assembly 55a. At this stage, the VU RF receiver 55c is waiting to receive a reset signal via the RF communication platform unit to trembling unit 96 (not shown in FIG. 8a, shown in FIG. 3). Once the reset signal is received, it signals the VU microcontroller 55b, which turns off the VU electronic adapter unit 58 and the VU trembling motor assembly 55a. It is also possible to have the VU turn itself off after a predefined period of time.

In order for the VU 50 to function properly, it includes a VU power supply 57 which is controlled by the VU power supply controller 56 for voltage stability, current drain and power consumption monitoring.

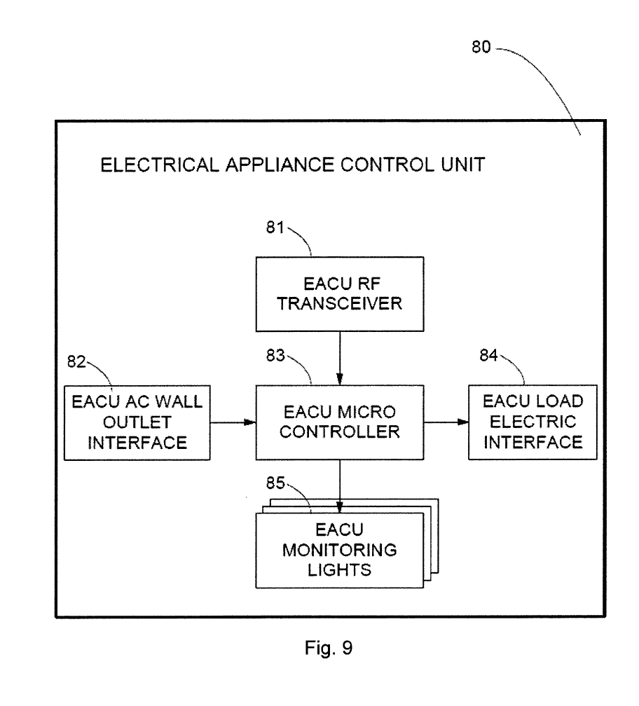

FIG. 9 is a block diagram of an electrical appliance control unit (EACU) 80, according to embodiments of the present invention. The EACU 80 connects to an AC wall outlet on the EACU AC wall outlet interface 82 and to an electrical appliance on the EACU load electrical interface 84. The EACU microcontroller 83 receives commands from the CU 20 (not shown in the present drawing), via the EACU RF transceiver 81, to allow electrical connection between the EACU AC wall outlet interface 82 and the EACU load electrical interface 84, providing electricity to the electrical appliance. The EACU monitoring lights 85 are used to indicate the status of the EACU 80.

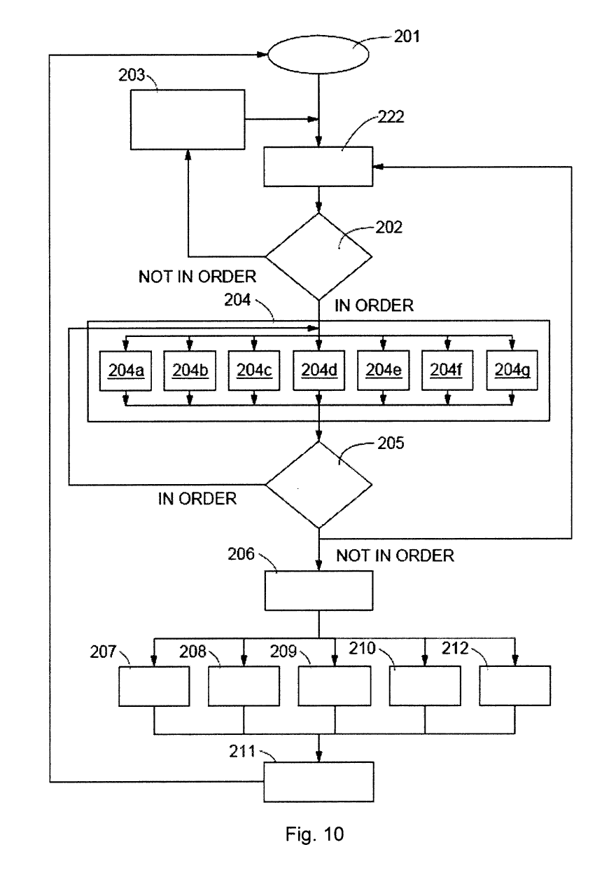

FIG. 10 is a flow chart that schematically illustrates a method for monitoring a baby's physiological conditions, for alerting, and for operating electrical appliances in accordance with an embodiment of the present invention.

Upon power up of the MAS 1, it enters the start stage (stage 201). Proper power up of the system includes first turning on the CU 20 and the PU 30, and receiving an indication from the CU 20 and PU 30 that they are functioning correctly (via their control lights or LCDs).

The first stage in the operation of the system, is the “measure air composition in the vicinity of the DU” (stage 222) where the DU 10 measures the different parameters of the air in the vicinity of the DU 10 such as, for example, the air humidity, temperature, CO₂, etc.

Immediately following that, the MAS 1 moves to the “test air composition in the vicinity of the DU” (stage 202) in which the DU 10, begins to test the air composition in its vicinity testing for hazardous materials such as carbon monoxide and cooking gas.

Should the air composition fail to meet the specified criteria for safety, namely ‘not in order’, the MAS 1, goes to “operate electrical appliances” (stage 203) in which the MAS 1, operates the EACU 80, that in turn operates the corresponding electrical appliance in order to influence the air composition. For example, the EACU 80, operates an electrical window opener to allow fresh air to enter the room.

In the case where the air composition test done in “test air composition in the vicinity of the DU” (stage 202) is within the specifications, namely ‘in order’ the system moves to the “measure breath parameters” stage (stage 204) where the DU 10, measures the baby's breath parameters such as movement of the lower abdomen Following that, the DU 10 should be turned on (by one of the implemented methods).

At this point, the DU 10 sounds (or transmits) a predefined sequence to the CU 20 for a communication check ensuring proper communications between the DU 10 and the CU 20.

The DU 10 then waits for several abdomen movements before moving into the full operative mode. This is done in order to prevent false alarms upon opening of the diaper 11 before placing it onto the baby.

Stage “measure breath parameters” (204) may be composed of several simultaneously occurring measurements such as “regular breath—Eupnea” (sub-stage 204a), “fibrillation” (sub-stage 204b), “respiratory arrest” (sub-stage 204c), “Dyspnea” (sub-stage 204d), “Bradypnea” (sub-stage 204e), “Hyperpnoea” and “Tachypnea” (sub-stage 204f) as well as a Built-In-Test (BIT), (sub-stage 204g). Thresholds and algorithms for detecting the various measurements are programmed into the CU microprocessor 21c.

After measuring the breath parameters, the DU 10, analyzes the measured breath parameters in “analyze breath parameters” (stage 205), and if the parameters are within the proper specification, the MAS 1, returns to “measure breath parameters” (stage 204) and continue to monitor the baby's breath parameters.

In the event that the breath parameters fall out of the proper specifications; the system moves into “initiate alert array” (stage 206).

In the “initiate DU alerts” (stage 207) where the DU sends a diaper unit to platform unit communication 91 signal in order for the CU 20 to send out the various alerts simultaneously: The “initiate crib vibrating” (stage 208) sends out the platform unit to vibrating unit RF communication 96 signal to initiate the VU 50 to vibrate the crib. The “initiate CU alerts” (stage 209) operates several alerts: an auditory alert using the CU loudspeaker 21a CU control light 26 and by sending the SMS message from platform unit to cellular telephone 92 and e-mail message 93.

The “initiate PU alerts” (stage 210) sends out the platform unit to parents' unit RF communication 94, in order to initiate the PU 30 alerts using the PU loudspeaker 31a and the PU liquid crystal display 31b.

The “initiate BIT alert” (stage 212) sends out a diaper unit to platform unit communication 91 in order to have the CU 20 sound an alert notifying that the BIT has failed.

Once the overseer (for example, parents) press the PU disable alarm button 35 or the CU disable alarm button 29 the system moves to the disable alerts (stage 211) where it turns off all of the alerts, and following that, restarts the system at the start (stage 201).

The software in the microcontroller has the ability to “learn” (with a proper algorithm) the typically abdomen movements for period of time. Thereafter, the initiation of an alert will be more accurate and stable, thereby eliminating false alarms. It means the DU may have the ability to adapt itself for each baby. This feature can be implemented in stage 204 as shown in FIG. 10.

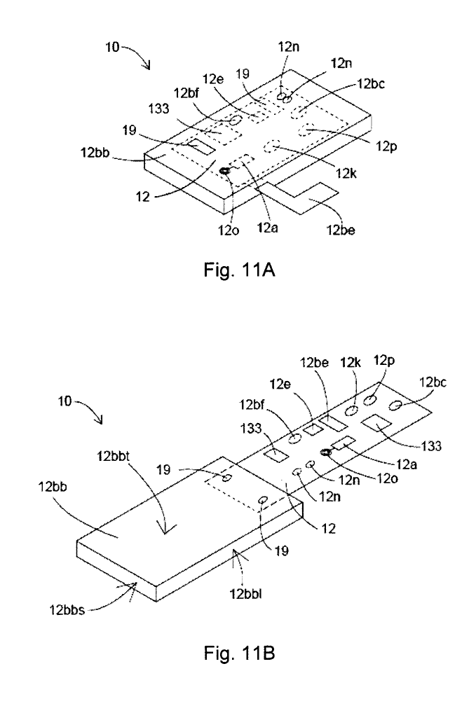

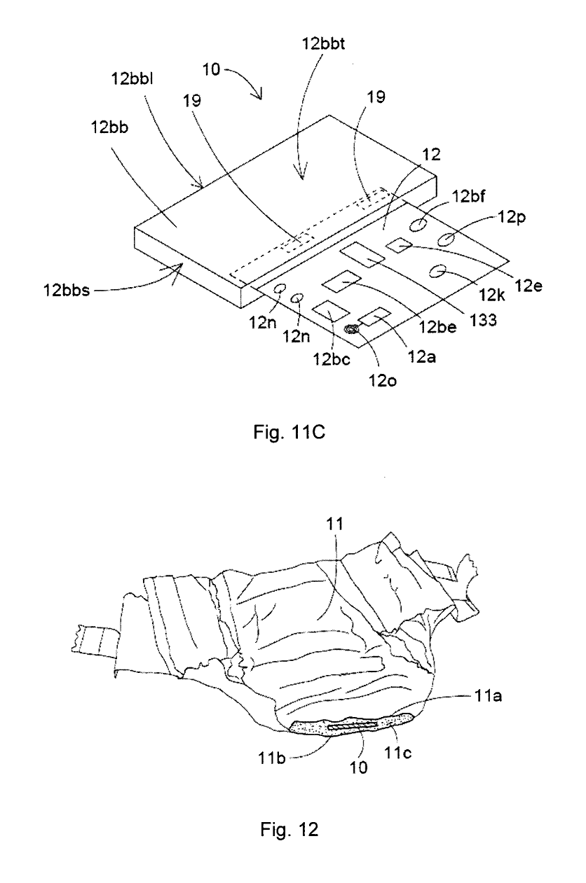

FIG. 11a is a diaper unit (DU) 10, according to embodiments of the present invention. The DU 10 includes a DU resistive sensor 12bb that is connected to a DU printed circuit board 12 which may be built into the DU resistive sensor 12bb itself or via two or more DU resistive sensor contacts 19. The DU printed circuit board 12 contains the required circuitry to communicate with the platform unit 20 (not shown in the present drawing), as well as the DU battery 12be, and DU power switch 12k and two DU discharge contacts 12n.

In this exemplary embodiment, the DU printed circuit board 12 is placed under or within the DU resistive sensor 12bb.

FIG. 11b is a diaper unit (DU) 10, which is disposable, according to embodiments of the present invention. In this exemplary embodiment, the DU printed circuit board 12 is connected to the DU resistive sensor top side 12bbt and is connected near the DU resistive sensor short side 12bbs and may be embedded within the DU resistive sensor 12bb itself.

FIG. 11c is a disposable diaper unit (DU) 10, which is disposable, according to embodiments of the present invention. In this exemplary embodiment, the DU printed circuit board 12 is connected to the DU resistive sensor top side 12bbt and is connected near the DU resistive sensor long side 12bbl and may be embedded within the DU resistive sensor 12bb itself.

FIG. 12 is a diaper 11 with an embedded DU resistive sensor 12bb in it, according to embodiments of the present invention. The DU resistive sensor 12bb can be manufactured into the diaper 11 between the diaper outer layer 11a and the diaper inner layer 11b, or it can be embedded within the diaper fluff material 11c so that the DU resistive sensor 12bb does not come in contact with the baby's skin.

FIG. 13a is a graph of experimental results of a DU resistive sensor 12bb in its idle state, according to some embodiments. In the idle state experiment, the DU resistive sensor 12bb (not shown in the present drawing), was attached to a diaper 11 (not shown in the present drawing), and placed on a table untouched. A resistance to voltage converter was connected to the DU resistive sensor 12bb (not shown in the present drawing), with its output captured by a Fluke 289 Data Logger. The output was sampled once every second for one hour. The voltage measured averaged at 0.574 Volts with a minimum value of 0.4884 Volts and a maximum value of 0.6348 Volts, with very little variance.

FIG. 13b is a graph of experimental results of a DU resistive sensor 12bb (not shown in the present drawing), in its active state, according to some embodiments. In the active state experiment, the same diaper 11 (not shown in the present drawing), used for the idle state experiment was put on a three week old baby and the sensors' output was captured once every second for one hour. The voltage measured averaged at 0.3812 Volts with a minimum value of 0.0508 Volts and a maximum value of 1.1033 Volts, with very high variance.

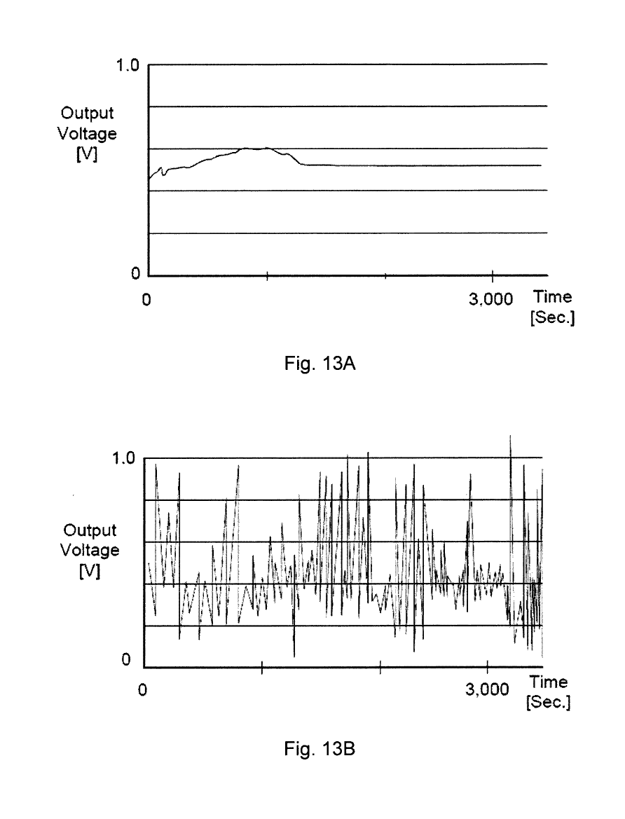

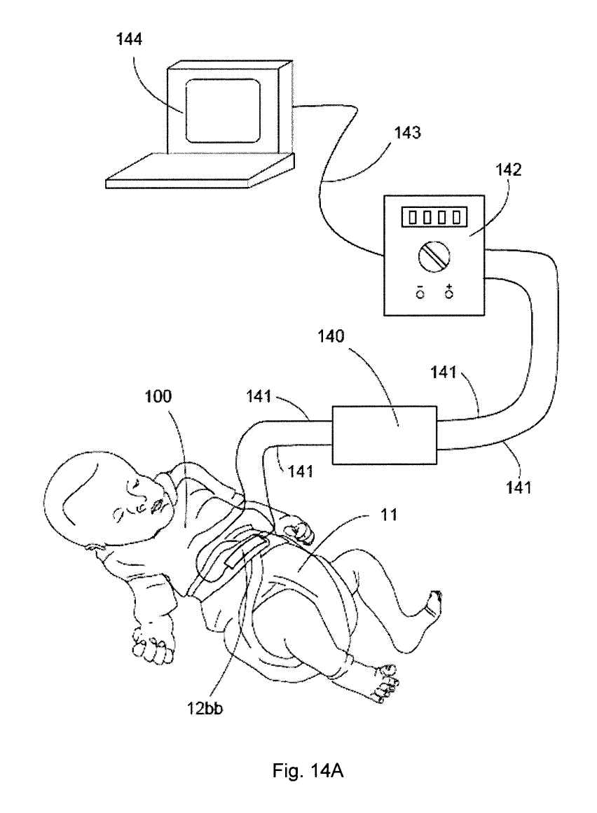

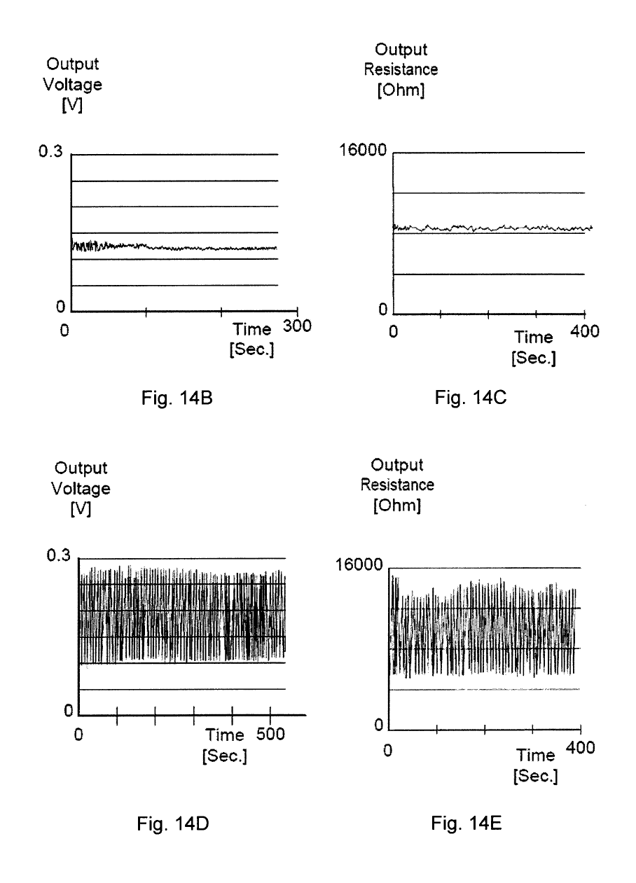

FIG. 14a is an experiment setup measuring various properties of a DU resistive sensor 12bb, according to embodiments of the present invention. In the active state experiments, a baby 100 was dressed with a diaper 11 equipped with a conductive fiber type DU resistive sensor 12bb. The DU resistive sensor 12bb was connected to a resistance to voltage converter 140 using a pair of conductive leads 141. A second pair of conductive leads 141 connects the resistance to voltage converter 140 to a data logger 142, which is connected to a personal computer (PC) 144 using a data logger to personal computer (PC) cable 143.

The resistance to voltage converter 140 is measuring the resistivity of the DU resistive sensor 12bb and converts it to a voltage value, which is logged by the data logger 142. The data logged by the data logger 142 is transferred to the PC 144 to be analyzed.

In order to test the resistivity of the DU resistive sensor 12bb, the resistance to voltage converter 140 is removed from the experiment setup and the DU resistive sensor 12bb is connected directly to the data logger 142.

The same experiments were also performed in an idle state, where the diaper 11 was laid stationary on a table instead of being worn by a baby 100.

FIG. 14b is a graph of the voltage measurements logged during an idle state experiment as described above.

In this experiment, data was logged every second for approximately 300 seconds. The voltage measured averaged at 0.1245 Volts with a minimum value of 0.1179 Volts and a maximum value of 0.1419 Volts, with very little variance.

FIG. 14c is a graph of the resistance measurements logged during an idle state experiment as described above.

In this experiment, data was logged every second for approximately 400 seconds. The voltage measured averaged at 8,590 Ohms with a minimum value of 8,262 Ohms and a maximum value of 8,956 Ohms, with very little variance.

FIG. 14d is a graph of the voltage measurements logged during an active state experiment as described above.

In this experiment, data was logged every second for approximately 600 seconds. The voltage measured averaged at 0.1661 Volts with a minimum value of 0.1016 Volts and a maximum value of 0.2934 Volts, with very high variance.

FIG. 14e is a graph of the resistance measurements logged during an active state experiment as described above.

In this experiment, data was logged every second for approximately 400 seconds. The voltage measured averaged at 10,234 Ohms with a minimum value of 5,089 Ohms and a maximum value of 15,315 Ohms, with very high variance.

According to these experiments, it is possible to define limits for deciding whether the sensor is sensing breath conditions or not, thus triggering an alert.

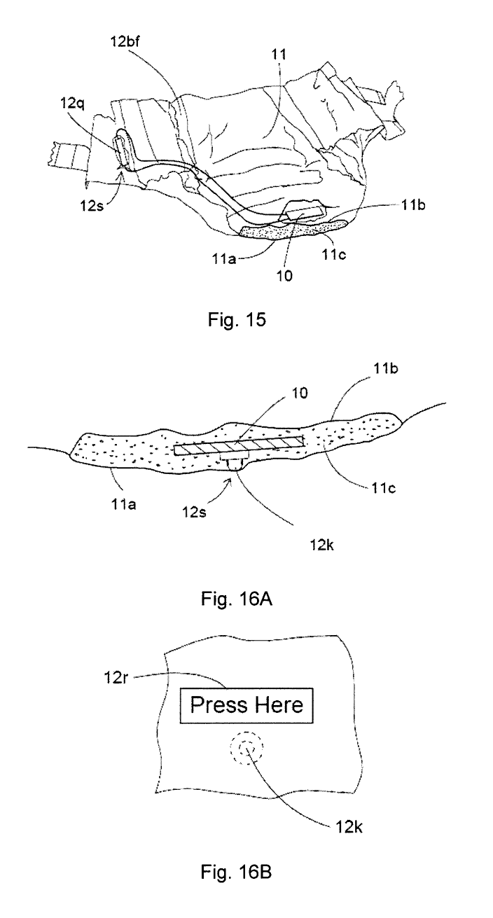

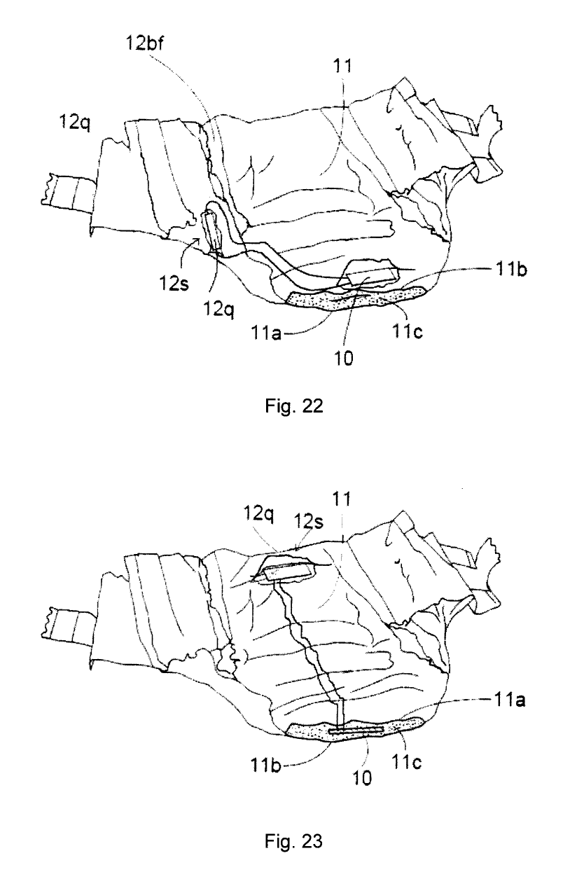

FIG. 15 is a diaper 11 equipped with a DU 10 with partial cutouts, and an activation mechanism 12s of a first type, according to an embodiment of the present invention. The partial cut outs show the DU 10 connected with a pair of DU conductive wires 12bf to a DU strain gauge 12q. It is possible to place the DU strain gauge 12q on the right side as well or to have two DU strain gauges 12q on both sides, and in any case, the present invention is in no way limited to any specific location on the diaper 11, or quantity, of the strain gauges 12q. FIG. 22 shows another position for the strain gauge. Another place is shown in FIG. 23, which is tight and shrinkable. When a person stretches this place, it is changing the strain gauge resistance.

The DU strain gauge 12q is used as the activation mechanism 12s. The DU strain gauge 12q is used as a power switch of the DU 10. When the diaper 11 is folded open, the DU strain gauge 12q is stretched and changes its resistivity thereby turning on the DU 10. Alternatively, the turning on mechanism may be implemented as a pull-strip (FIG. 25).

In this way, the user does not need to perform any additional actions in order to turn on the DU 10. The DU strain gauge 12q can be made out of flexible materials such as an elastomer or a conductive wire.

FIG. 16a is a front view of a diaper 11 equipped with a DU 10, and a DU activation mechanism 12s of another type, according to an embodiment of the present invention. It shows the DU 10 equipped with a DU power switch 12k which in this case, made from a flexible material (such as silicon rubber) and is placed just under the diaper outer layer 11a. The power switch 12k is used as the DU activation mechanism 12s.

FIG. 16b is a bottom view of a diaper 11 equipped with a DU 10, and a DU activation mechanism 12s of a second type, according to an embodiment of the present invention.

FIG. 16b shows a DU label 12r showing a message “Press Here” located over the DU power switch 12k as described in FIG. 16a. The label instructs the user on the correct location to press in order to activate the DU 10. The label may be written in other languages as well, contain a graphic symbol, or a combination of both.

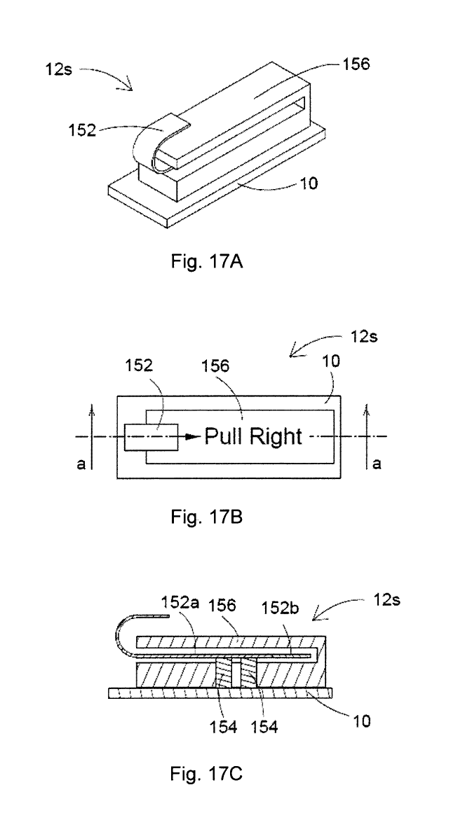

FIG. 17a is a DU 10 equipped with a pull strip assembly 156, as a DU activation mechanism 12s of a third type, according to an embodiment of the present invention. The pull strip assembly 156 is affixed on top of the DU 10 and is used to activate the DU 10 by pulling the pull strip 152, or the pull strip assembly 156 is affixed between the diaper outer layer 11a and the diaper inner layer 11b in DU 10 and is used to activate the DU 10 by pulling fastening flap 11d which connected to pull strip 152.

FIG. 17b is a top view, schematic illustration of an illustrative, DU 10 equipped with a pull strip assembly 156, as a DU activation mechanism 12s of another type, upon the section plane a-a is marked, according to an embodiment of the present invention.

FIG. 17c is a cross sectional side view a-a of illustrative embodiment of a DU 10 equipped with a pull strip assembly 156, as a DU activation mechanism 12s of another type, according to embodiments of the present invention. The pull strip 152 is composed of two sections: a non-conductive portion of pull strip 152a and a conductive portion of pull strip 152b. By pulling the protruding part of the pull strip 152, the non-conductive portion of pull strip 152a pulls the conductive portion of pull strip 152b until it moves over the two pull strip case contacts 154, thereby closing an electrical circuit and switching on the DU 10. At this stage, the non-conductive portion of pull strip 152a is pulled apart of the conductive portion of pull strip 152b and is discarded.

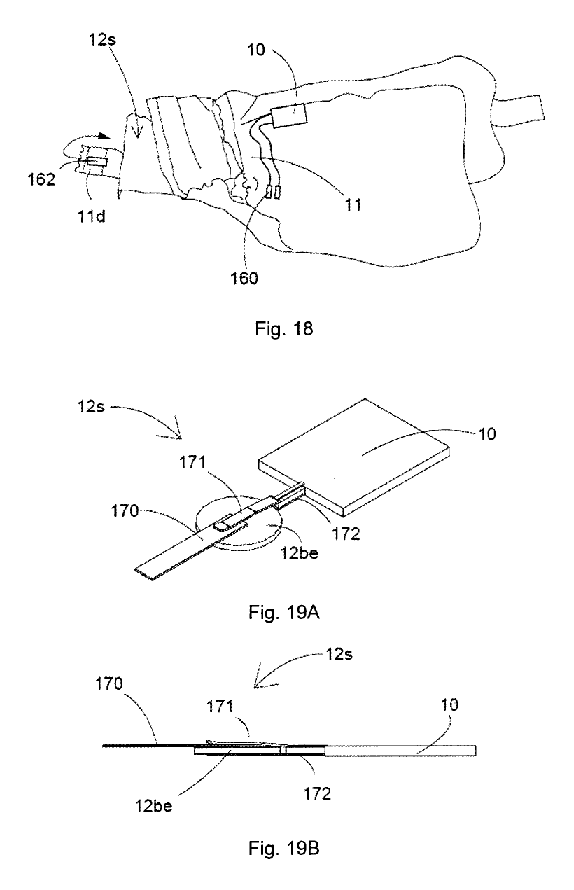

FIG. 18 shows a diaper 11 equipped with a DU 10, and a DU activation mechanism 12s of another type, according to embodiments of the present invention. When the DU 10 is used in the present figure's configuration, the diaper fastening flap 11d is used to fasten the diaper 11 as with a regular diaper 11 except that when the diaper fastening flap 11d is fastened, the DU conductive patch 162 (which is placed on the inner side of the diaper, fastening flap 11d) comes in contact with the two DU power contacts 160, closes an electrical circuit, and turns on the DU 10.

FIG. 19a is a DU 10, and a DU activation mechanism 12s, according to an embodiment of the present invention. The DU 10 is connected to a DU battery 12be using a first battery contact 171 and a second battery contact 172. A non-conductive pull strip 170 is kept between the first battery contact 171 and the DU battery 12be so that there is no electrical contact between them and the electrical circuit is open. Only when the user removes the non-conductive pull strip 170, does the DU 10 turn on. FIG. 19b is a DU 10, and a DU activation mechanism 12s of a fifth type, according to an embodiment of the present invention as presented in FIG. 19a.

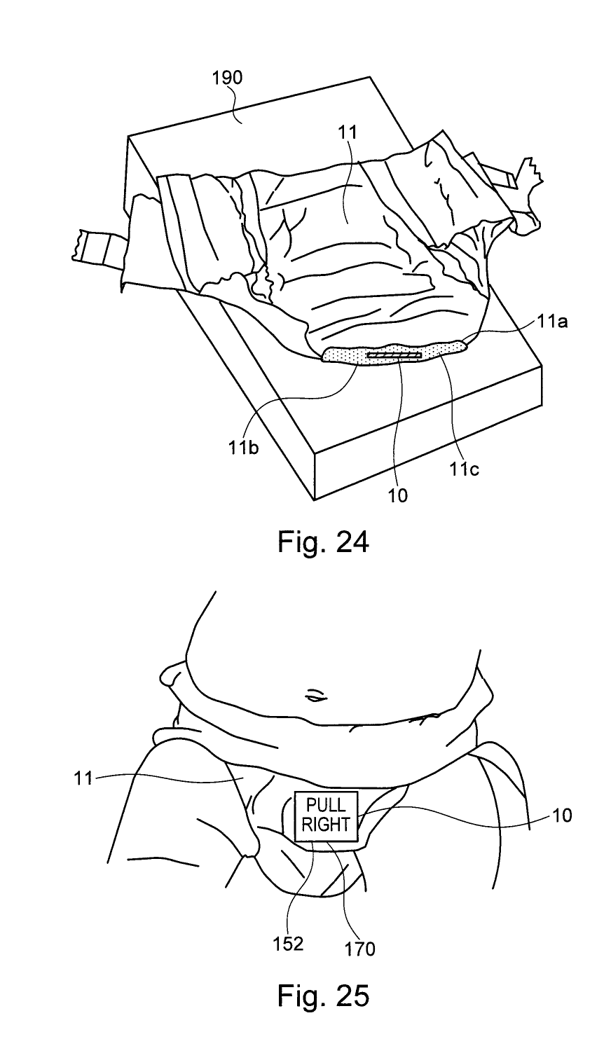

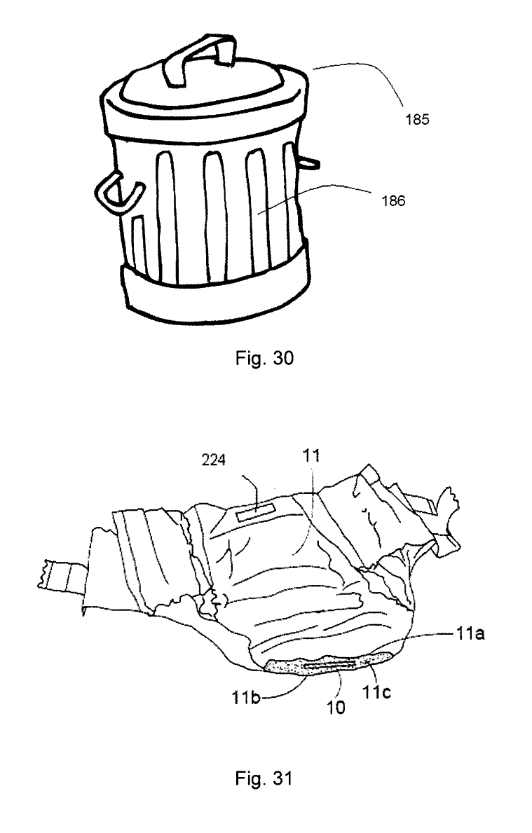

Various other known methods may be used for activating the battery and the DU 10. These various implementations include, for example, but not limited to: reed switches, magnetic strip 224 (FIG. 31) (e.g. when the diaper is opened then a reed switch on the sensor side is switched on (N.O. type) because the magnetic field is gone), built in activation mechanism in a battery with reed switch or pull strip (FIG. 17a), cover removal 223 for activating Zinc-Air battery (FIG. 21), two discharge terminals that will short circuit by conductive label (FIG. 29), discharge by reed switch and magnetic tray (FIG. 24) or trash can (FIG. 30), and discharge by magnetic field.

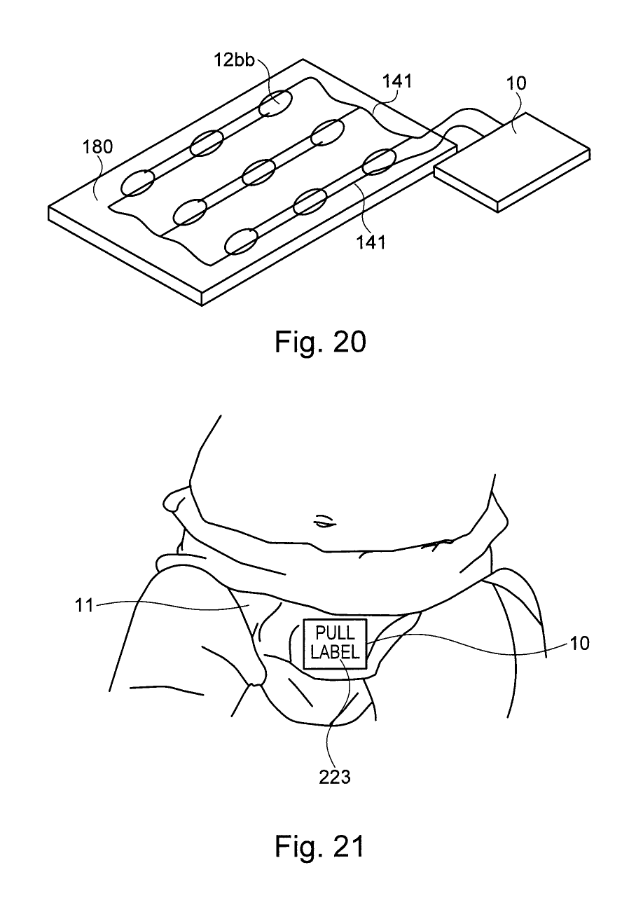

FIG. 20 is a diaper unit 10, connected to an array of DU resistive sensors 12bb laid out in a grid onto a resistive sensor support sheet 180 using a set of conductive leads 141, according to an embodiment of the present invention. The resistive sensor support sheet 180 may be embedded within the cribs' mattress or within the mattress' sheets.

The same principles apply to the case where the person being monitored is not a baby and sleeps in a regular bed. It is easy to implement the resistive sensor support sheet 180 in any standard mattress and bed sheet.

The various embodiments herein described involve several creative, surprising and unique features. One such aspect is fitting the electronic diaper mechanism as an integral part of the diaper—the mechanism is inserted directly into the diaper with the sensor as part of the manufacturing process (built in). Another such feature is automatic operation of the baby diaper mechanism—activation of the diaper mechanism upon opening of the diaper without any user particular activity. Unique to this disclosure is the use of sensors not previously used for sensing the physiological conditions of a body. The sensor may also be implemented as a sticker and placed anywhere on the body or in the diaper. Depending on the embodiment, the sensor may be an integral part of the electronic circuit or be made separate. To implement such embodiments, there was necessarily unique ASIC development only for this use. There is acoustic communication between the baby diaper mechanism to the in-platform unit for maintenance—acoustical communication for alerting about physiologic problems or diaper mechanism failure, or proper function indication in any known audible or non-audible sound interval. Moreover, the system influences the baby environment in order to wake him up—operating appliance to vibrate the crib. The subject unit and platform unit can be used in various places outside the baby bed, for example, as part of/associated with/integrated with/attached to a baby carriage or stroller, infant seat, crib, and the like. Further application of the system is for bedclothes—the mechanism can be embedded inside baby mattress or bed sheet with elastomeric/piezo/resistive sheet/conductive fabric which serve as the sensor device. There can be discharge of the diaper battery by: placement on a magnetic plate or trash can and activation of the diaper reed switch; automatic activation of the load resistor by the microcontroller after a period of non movement of the sensor; or by using a wireless signal. One embodiment has an embedded operating mechanism inside a battery. The platform unit also monitors the environment. There are various types of communications for alerting a responsible person—SMS, windows application, email.

While certain features of the invention have been illustrated and described herein, many modifications, substitutions, changes, and equivalents will now occur to those of ordinary skill in the art. The invention is described in detail with reference to a particular embodiment, but it should be understood that various other modifications can be effected and still be within the spirit and scope of the invention. It is, therefore, to be understood that the appended claims are intended to cover all such modifications and changes as fall within the true spirit of the invention.

In the description and claims of the application, each of the words “comprise” “include” and “have”, and forms thereof, are not necessarily limited to members in a list with which the words may be associated.

1. A system for monitoring breathing of a subject, the system comprising:
(a) a wearable subject unit comprising:
an elastic resistive sensor positionable such that breathing motion of the subject applies mechanical pressure to said elastic resistive sensor, wherein said elastic resistive sensor is configured to change its resistance responsive to the mechanical pressure, and
a transmitter configured to wirelessly transmit a signal based on the change in resistance of the elastic resistive sensor; and
(b) a platform unit comprising a receiver and being positionable in wireless transmission range with said subject unit, said platform unit configured to receive said signal from said subject unit and to issue an advisory signal indicative of the breathing of the subject.

2. The system according to claim 1, wherein said elastic resistive sensor comprises an elastic, electrically-conductive material contained within a non-electrically-conductive housing.

3. The system according to claim 2, wherein said elastic, electrically-conductive material is selected from the group consisting of: foam soaked in a conductive electrolyte, a conductive fabric, a conductive paper, conductive paint, conductive ink, a conductive elastomer and a piezoresistive sheet.

4. The system according to claim 1, wherein said wearable subject unit is embedded within a disposable diaper.

5. The system according to claim 1, wherein said transmitter of said wearable subject unit is a radio frequency (RF) transmitter, and said receiver of said platform unit is an RF receiver.

6. The system according to claim 1, wherein said transmitter of said wearable subject unit is an acoustic transmitter, and said receiver of said platform unit is an acoustic receiver

7. The system according to claim 1, further comprising a remote unit configured to receive said advisory signal from said platform unit and to issue an alert to a supervisor of the subject.

8. The system according to claim 1, further comprising a tactile unit configured to reinvigorate said subject based on the signal from said wearable subject unit, when the signal indicates lack of breathing or breathing rate below a predetermined threshold.

9. The system according to claim 1, further comprising an environmental detector configured to detect at least one environmental parameter potentially affecting the breathing of the subject, the at least one environmental parameter selected from the group consisting of:
temperature, humidity, light and carbon monoxide.

10. The system according to claim 9, further comprising an interface unit configured to affect the at least one environmental parameter in order to improve the breathing of the subject.

11. The system according to claim 1, further comprising an activator for electrically activating said elastic resistive sensor, said activator is selected from the group consisting of: a strain gauge, a pull strip, a circuit-shortening conductive patch, a non-conductive pull strip on a battery contact, a reed switch, a battery comprising a built-in activation mechanism and a cover for activating a Zinc-air battery.

12. A disposable diaper comprising:
an elastic resistive sensor embedded within said disposable diaper and being positionable such that breathing motion of a subject wearing the disposable diaper applies mechanical pressure to said elastic resistive sensor, wherein said elastic resistive sensor is configured to change its resistance responsive to the mechanical pressure; and
a transmitter embedded within said disposable diaper and being configured to wirelessly transmit a signal based on the change in resistance of the elastic resistive sensor.

13. The disposable diaper according to claim 12, wherein said elastic resistive sensor comprises an elastic, electrically-conductive material contained within a non-electrically-conductive housing.

14. The disposable diaper according to claim 13, wherein said elastic, electrically-conductive material is selected from the group consisting of: foam soaked in a conductive electrolyte, a conductive fabric, a conductive paper, conductive paint, conductive ink, a conductive elastomer and a piezoresistive sheet.

15. The disposable diaper according to claim 12, wherein said transmitter is a radio frequency (RF) transmitter.

16. The disposable diaper according to claim 12, wherein said transmitter is an acoustic transmitter.

17. A disposable diaper comprising:
an elastic resistive sensor embedded within said disposable diaper and being positionable such that breathing motion of a subject wearing the disposable diaper applies mechanical pressure to said elastic resistive sensor, wherein said elastic resistive sensor is configured to change its resistance responsive to the mechanical pressure;
a transmitter embedded within said disposable diaper and being configured to wirelessly transmit a signal based on the change in resistance of the elastic resistive sensor; and
an activator for electrically activating said elastic resistive sensor.

18. The disposable diaper according to claim 17, wherein said activator is selected from the group consisting of: a strain gauge, a pull strip, a circuit-shortening conductive patch, a non-conductive pull strip on a battery contact, a reed switch, a battery comprising a built-in activation mechanism and a cover for activating a Zinc-air battery.

19. The disposable diaper according to claim 17, wherein said elastic resistive sensor comprises an elastic, electrically-conductive material contained within a non-electrically-conductive housing.

20. The disposable diaper according to claim 19, wherein said elastic, electrically-conductive material is selected from the group consisting of: foam soaked in a conductive electrolyte, a conductive fabric, a conductive paper, conductive paint, conductive ink, a conductive elastomer and a piezoresistive sheet.

21-22. (canceled)
